(19) United States
(12) Patent Application Publication
(10) Pub. No.: US 2007/0196334 A1
(43) Pub. Date: Aug. 23, 2007

(54) Modified vectors for organelle transfection

(75) Inventor: Shaharyar Khan, North Garden, VA (US)

Correspondence Address: PATREA L. PABST;PABST PATENT GROUP LLP, 400 COLONY SQUARE, SUITE 1200, 1201 PEACHTREE STREET, ATLANTA, GA, 30361, US

(73) Assignees: Shaharyar Khan; Gencia Corporation

(21) Appl. No.: 10/561,829

(22) PCT Filed: Jun. 25, 2004
(86) PCT No.: PCT/US04/20454

§ 371 Date: Mar. 23, 2007

Related U.S. Application Data
(60) Provisional application No. 60/482,603, filed on Jun. 25, 2003.

(51) Int. Cl. A61K 48/00; C12N 9/22; C12N 15/86; C12N 15/74; C12N 5/10

(52) U.S. Cl. 424/93.2; 435/456; 435/472; 435/199; 435/252.3; 435/325; 977/802

(57) ABSTRACT

The present disclosure provides compositions and methods for direct transfection of organelle DNA in living cells. More particularly, the present disclosure is based on the use of viral vectors that contain localization signals specific for the target organelle. In one embodiment a viral vector that has been modified to express on its surface a protein transduction domain and an organelle localization signal is provided. A viral vector comprising a desired recombinant DNA construct is introduced into cell culture or injected into an organism, wherein the viral vector transduces across the cellular membrane through its protein transduction domain and localizes to the cellular organelle by way of the organelle localization/targeting signal introducing the recombinant DNA into the interior of the organelle

### CROSS REFERENCE TO RELATED APPLICATION

This application claims benefit of and priority to U.S. Provisional Patent Application No. 60/482,603 filed on Jun. 25, 2003, which is incorporated by reference herein in its entirety.

### INCORPORATION-BY-REFERENCE

This application incorporates by reference the sequence listing on the accompanying compact disc in its entirety. The filename for the sequence listing is 120701-8010.ST25.txt which was created on Jun. 23, 2004, and is about 772 KB.

### BACKGROUND

1. Field of the Disclosure

The present disclosure is generally directed to compositions and methods for transfecting cells and organelles, in particular modified viral vectors for transfecting mitochondria and chloroplasts.

2. Related Art

Mitochondria are the sole energy-producing organelles in all eukaryotic cells, and therefore play a critical role in maintaining proper cellular bioenergetics, homeostatic levels and cellular life cycles. Similarly, chloroplasts are also efficient ATP-producing machines that use light as the source of energy rather than sugars or fatty acids. Both mitochondria and chloroplasts contain multiple copies of organelle DNA that is replicated and transcribed in the organelles. In mammals, mitochondrial DNA (mtDNA) is a circular, approximately 16.5 kilobase, intronless genome that encodes 13 electron transport chain (ETC) proteins, 2 ribosomal RNA's and 22 tRNA's. Chloroplast genomes range in size from 40-150 kilobases. Most insights into mitochondrial genetics have come in yeast, where biolistic transformation allows for engineering of mitochondrial replicons. However, many features of mammalian mitochondrial gene expression and respiratory chain biogenesis are not reproducible in yeast.

In mammals, cytoplasmic fusion and microinjection are used to introduce donor mitochondria, but these techniques fail to provide a mechanism for the direct manipulation of mtDNA. In addition the uptake of exogenous DNA into mitochondria involving the protein import pathway has been reported from two laboratories. Vestweber and Schatz (1989) Nature (London) 338:170-172 achieved uptake of a 24-bp both single- and double-stranded oligonucleotide into yeast mitochondria by coupling the 5′ end of the oligonucleotide to a precursor protein consisting of the yeast cytochrome c oxidase subunit IV presequence fused to a modified mouse dihydrofolate reductase. More recently, Seibel et al. (1995) Nucleic Acids Research 23:10-17 reported the import into the mitochondrial matrix of double-stranded DNA molecules conjugated to the amino-terminal leader peptide of the rat ornithine-transcarbamylase. Both studies, however, were done with isolated mitochondria, not addressing the question of how oligonucleotide-peptide conjugates will pass the cytosolic membrane and reach mitochondrial proximity.

U.S. Pat. No. 6,171,863 discloses the use of dequalinium-DNA complexes as a vehicle for delivering DNA to the interior of cells and potentially to the mitochondria. Because the DNA is associated with dequalinium, the resulting complex has a positive charge. The positively charged complex is attracted to negatively charged compartments. Thus, U.S. Pat. No. 6,171,863 discloses delivery of DNA to negatively charged compartments, and does not disclose the specific delivery DNA to mitochondria or chloroplasts. Indeed, no technique has been disclosed for targeting specific organelles, for example the chloroplast or mitochondria, for the delivery of nucleic acids using a receptor-independent mechanism.

Thus, the inability to specifically manipulate the chloroplast and mitochondrial genome has hampered researchers' efforts to fully understand chloroplast and mtDNA replication, transcription, and translation processes. The ability to specifically manipulate mtDNA and introduce it into living cells would greatly enhance researchers' ability to fully investigate the function of individual chloroplast/mitochondrial genes and overall chloroplast/mitochondrial function.

Furthermore, the ability to manipulate the mitochondrial genome also provides a novel method of treating diseases associated with defective mitochondrial function. With age, the function of mitochondria decreases with a marked increase of mutations and large deletions of mtDNA. In particular, oxidative damage increases with age, often leading to a higher rate of mtDNA mutations. Aside from known mtDNA mutations, several forms of cancer and neurodegeneration are associated with mutations in mtDNA. For example, mutations in mitochondrial DNA are the suspected cause of a host of degenerative neurological diseases including Alzheimers, Parkinsons and adult-onset diabetes. These mutations result in decreased electron transport chain efficiency, and the build-up of mtDNA deletions due to free radical damage (aging).

In addition, given the bioenergetic functions of chloroplasts, the ability to introduce exogenous genes or otherwise manipulate the chloroplast genome could have a tremendous impact on increasing the vitality and yields of crops and other plants. For example, introduction of genes into chloroplast may lead to plants with increased viability in otherwise hostile environments and increased efficiency of photosynthesis. In addition, the expression of exogenous genes within the chloroplasts is believed to be significantly more efficient in chloroplasts relative the expression of exogenous genes introduced into the nucleus of the cell. Thus transfection of chloroplasts may allow for more effective biosynthesis strategies for commercial compounds.

In light of the numerous disease conditions related to organelle dysfunction, there is a need for methods and compositions for treating such disease conditions. Accordingly, there is also a need for improved methods and compositions for introducing polynucleotides into specific organelles.

There is also a need for methods of treating diseases related to organelle dysfunction including targeting polynucleotides to dysfunctional organelles or cells.

### SUMMARY OF THE DISCLOSURE

The present disclosure is generally directed to compositions, methods and systems for introducing polynucleotides into an cell or organelle of a cell, for example a eukaryotic cell. One aspect of the present disclosure provides nucleic acid constructs and methods for delivering nucleic acids to specific organelles. The targeting of polynucleotides to specific organelles can be accomplished without using receptor mediated localization techniques. Receptor mediated localization techniques means techniques in which the polynucleotide construct displays a ligand or receptor that is recognized by its complement on a specific organelle. A particular aspect of the disclosure provides methods and compositions for transfecting organelles by incorporating a protein transduction domain (PTD) in combination with an organelle localization/targeting signal on a vector, for example a viral vector. In one aspect of the disclosure, targeting signals do not act through a receptor:ligand interaction. Typically, the modified virus expresses both a protein transduction domain as well as an organelle localization/targeting signal that can associate with a specific organelle. Suitable viral vectors include but are not limited to a viral vector such as bacteriophage lambda. Exemplary PTDs include but are not limited to HIV TAT YGRKKRRQRRR (SEQ. ID NO. 3) or RKKRRQRRR (SEQ. ID NO. 4); 11 Arginine residues, or positively charged polypeptides or polynucleotides having 8-15 residues, preferably 9-11 residues. Exemplary organelle localization signals include but are not limited to those listed in Tables 1 and 2.

Another aspect of the disclosure provides a system including an intact viable cell or organism that contains a recombinant vector. The recombinant vector can specifically cross the plasma membrane of the organism or cell and can localize/target to a specific organelle. The cell or organism can further contain a genome modified by the recombinant vector.

Other aspects of the disclosure provide methods of correcting polynucleotide defects, including heritable polynucleotide defects or acquired polynucleotide defects, augmenting expression of specific nucleic acids, interfering with the expression of specific nucleic acids, restoring or augmenting organelle function, increasing biosynthesis of specific nucleic acids and their corresponding proteins using targeted delivery of nucleic acids to specific cellular organelles or compartments.

Still other aspects of the present disclosure are directed to minimizing or reducing disease progression, alleviating symptoms, and adjusting cellular metabolism by transfecting specific organelles. Particular aspects are directed targeted delivery of nucleic acids to organelles containing the components for replication, transcription, or translation, or a combination thereof such as the mitochondrion or chloroplast.

Another aspect of the disclosure provides compositions and methods for producing cell lines with depleted mitochondrial DNA.

### BRIEF DESCRIPTION OF THE DRAWINGS

FIG. 1A is a diagram of a modified bacteriophage lambda expressing a fusion protein of Head protein gpD including a Protein Transduction Domain, an organelle targeting signal, and a reporter protein (Red Fluorescent Protein).

FIG. 1B is a diagram of the 1185 nucleotide construct PTD-MLR-gpD encoding a Protein Transduction Domain, Mitochondrial Targeting Signal, Red Fluorescent Protein fusion with gene product D.

FIG. 1C is a diagram of plasmid pEXP-TMRD.

FIG. 2A is a diagram of a full-length mtDNA PCR amplicon generated with sense and anti-sense primers containing internal BgIII and NotI sites, respectively.

FIG. 2B is a diagram of a full length mtDNA amplicon ligated to Green Fluorescent Protein (GFP) DNA.

FIG. 3 is a diagram of full length mtDNA/GFP ligated to SuperCos-1.

FIG. 4 is a panel of confocal fluorescence micrographs showing Mitochondrial Targeting Signal with Red Fluorescent Protein and Protein Transduction Domain Containing Bacteriophage Lambda colocalizing with mitochondria in Sy5y cells in culture over a 40 minute time period. The 40-minute micrograph includes mitochondria specific dye, Mitotracker Green (Molecular Probes) to verify mitochondrial localization.

FIGS. 5A and 5B are Western Blots mitochondrial fractions of transfected cells generated at specific time points using antibodies to Red Fluorescent Protein (RFP).

FIG. 6A is a gel showing GFP message detected in mitochondrial fractions.

FIG. 6B is a collection of panels (a)-(d). Panels (a)-(c) are confocal fluorescence micrographs of rho⁰ cells (a) 24 hours after transfection with RFP recombinant phage; (b) initially transfected with pMLS-LambdaR (no RFP) following second transfection with SuperCos-1/mtDNA/GFP cosmid; and (c) companion images of MitoTracker Red staining (c) to reveal location of mitochondria. Panel (d) is a scatterplot comparing fluorescence intensities among MitoTracker Red mitochondrial clusters and GFP reporter gene expression.

FIG. 7 is a histogram showing dsRNA knockdown of POlG.

### DETAILED DESCRIPTION OF THE DISCLOSURE

1. Definitions

In describing and claiming the disclosure, the following terminology will be used in accordance with the definitions set forth below.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “treating” includes alleviating the symptoms associated with a specific disorder or condition and/or preventing or eliminating said symptoms.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will direct the linked protein to be localized at the specific organelle.

“Organelle Localization Signal” or “Organelle Targeting Signal” are used interchangeably and refer to a signal that directs a molecule to a specific organelle. The signal can be polynucleotide or polypeptide signal, or can be an organic or inorganic compound sufficient to direct an attached molecule to a desired organelle. Exemplary organelle localization signals are provided in Tables 1 and 2 and described in Emanuelson et al., Predicting Subcellular Localization of Proteins Based on Their N-terminal Amino Acid Sequence. Journal of Molecular Biology. 300(4):1005-16, 2000 Jul. 21, and in Cline and Henry, Import and Routing of Nucleus-encoded Chloroplast Proteins. Annual Review of Cell & Developmental Biology. 12:1-26, 1996, the disclosures of which are incorporated herein by reference in their entirety. It will be appreciated that the entire sequence listed in Tables 1 and 2 need not be included, and modifications including truncations of these sequences are within the scope of the disclosure provided the sequences operated to direct a linked molecule to a specific organelle. Organelle localization signals of the present disclosure can have 80 to 100% homology to the sequences in Tables 1 and 2. Suitable organelle localization signals include those that do not interact with the targeted organelle in a receptor:ligand mechanism. For example, organelle localization signals include signals having or conferring a net charge, for example a positive charge. Positively charged signals can be used to target negatively charged organelles such as the mitochondria. Negatively charged signals can be used to target positively charged organelles.

“Protein Transduction Domain” or PTD refers to a polypeptide, polynucleotide, or organic or inorganic compounds that facilitates traversing a lipid bilayer, micelle, cell membrane, organelle membrane, or vesicle membrane. A PTD attached to another molecule facilitates the molecule traversing membranes, for example going from extracellular space to intracellular space, or cytosol to within an organelle. Exemplary PTDs include but are not limited to HIV TAT YGRKKRRQRRR (SEQ. ID NO. 3) or RKKRRQRRR (SEQ. ID NO. 4); 11 Arginine residues, or positively charged polypeptides or polynucleotides having 8-15 residues, preferably 9-11 residues.

As used herein, the term “exogenous DNA” or “exogenous nucleic acid sequence” refers to a nucleic acid sequence that was introduced into a cell or organelle from an external source. Typically the introduced exogenous sequence is a recombinant sequence.

As used herein, the term “transfection” refers to the introduction of a nucleic acid sequence into the interior of a membrane enclosed space of a living cell, including introduction of the nucleic acid sequence into the cytosol of a cell as well as the interior space of a mitochondria, nucleus or chloroplast. The nucleic acid may be in the form of naked DNA or RNA, associated with various proteins or the nucleic acid may be incorporated into a vector.

As used herein, the term “vector” is used in reference to a vehicle used to introduce a nucleic acid sequence into a cell. A viral vector is virus that has been modified to allow recombinant DNA sequences to be introduced into host cells or cell organelles.

As used herein, the term “organelle” refers to cellular membrane bound structures such as the chloroplast, mitochondrion, and nucleus. The term “organelle” includes natural and synthetic organelles.

As used herein, the term “non-nuclear organelle” refers to any cellular membrane bound structure present in a cell, except the nucleus.

As used herein, the term “polynucleotide” generally refers to any polyribonucleotide or polydeoxribonucleotide, which may be unmodified RNA or DNA or modified RNA or DNA. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising. DNA and RNA that may be single-stranded or, more typically, double-stranded or a mixture of single- and double-stranded regions. The term “nucleic acid” or “nucleic acid sequence” also encompasses a polynucleotide as defined above.

In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide.

As used herein, the term polynucleotide includes DNAs or RNAs as described above that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

Oligonucleotides refers to relatively short polynucleotides. Often the term refers to single-stranded deoxyribonucleotides, but it can refer as well to single-or double-stranded ribonucleotides, RNA:DNA hybrids and double-stranded DNAs, among others.

As used herein the term “vector” means a polynucleotide molecule originating from a virus, a plasmid, or the cell of a higher organism into which another polynucleotide fragment of appropriate size can be integrated without loss of the vectors capacity for self-replication. Vectors introduce foreign or exogenous DNA into host cells, where it can be reproduced in large quantities. Examples are plasmids, cosmids, lambda phage vectors, P1 bacteriophage vectors, yeast artificial chromosomes, and mammalian artificial chromosomes. Vectors are often recombinant molecules containing nucleotide sequences sequences from several sources.

2. Transfection Compositions

The provided compositions include a polynucleotide vector operably linked to a protein transduction domain and a targeting signal. One embodiment provides a vector having a polynucleotide encoding a protein transduction domain operably linked to a targeting signal, wherein the protein transduction domain operably linked to the targeting signal is displayed on an exterior surface of the vector. The disclosed compositions and methods are useful for the transfection of eukaryotic cells and organelles, in particular mammalian cells and organelles. Cellular organelles have significant roles in the life cycle of cells and their hosts. For example, mitochondria and chloroplasts are the “powerhouses” of animal and plant cells. Because of their critical activity in maintaining cell life and bioenergetics, they play major roles in cell function and cell death. They possess their own unique genomes—which, until now, have remained unsuited to manipulation. Accordingly, some embodiments of the present disclosure provide compositions and methods for the delivery of a polynucleotide to a specific organelle, for example to mitochondria and chloroplasts. Delivered polynucleotides can encode a functional polypeptide that can be expressed in the organelle. Expression of the polypeptide in the organelle can modulate the function of the organelle and thereby alleviate symptoms of disease related to organelle dysfunction. In one embodiment, the mitochondrial genome (SEQ. ID. NO. 6) can be transfected into an organelle, either in whole or part. In other embodiments, the polynucleotide can encode an anti-sense polynucleotide or an enzymatic polynucleotide, including, but not limited to, a DNAzyme or ribozyme.

2.1 Organelles

Other embodiments of the present disclosure are directed to specifically delivering polynucleotides to cellular compartments or organelles. The polynucleotides can encode a polypeptide or interfere with the expression of a different polynucleotide. Eukaryotic cells contain membrane bound structures or organelles. Organelles can have single or multiple membranes and exist in both plant and animal cells. Depending on the function of the organelle, the organelle can consist of specific components such as proteins and cofactors. The polynucleotides delivered to the organelle can encode polypeptides that can enhance or contribute to the functioning of the organelle. Some organelles, such as mitochondria and chloroplasts, contain their own genome. Nucleic acids are replicated, transcribed, and translated within these organelles. Proteins are imported and metabolites are exported. Thus, there is an exchange of material across the membranes of organelles. In some embodiments, polynucleotides encoding mitochondrial polypeptides are specifically delivered to mitochondria.

Exemplary organelles include the nucleus, mitochondrion, chloroplast, lysosome, peroxisome, Golgi, endoplasmic reticulum, and nucleolus. Synthetic organelles can be formed from lipids and can contain specific proteins within the lipid membranes. Additionally, the content of synthetic organelles can be manipulated to contain components for the translation of nucleic acids.

2.1.1 Mitochondria

In other embodiments of the present disclosure, modified vectors are disclosed that specifically deliver polynucleotides to mitochondria. Mitochondria contain the molecular machinery for the conversion of energy from the breakdown of glucose into adenosine triphosphate (ATP). The energy stored in the high energy phosphate bonds of ATP is then available to power cellular functions. Mitochondria are mostly protein, but some lipid, DNA and RNA are present. These generally spherical organelles have an outer membrane surrounding an inner membrane that folds (cristae) into a scaffolding for oxidative phosphorylation and electron transport enzymes. Most mitochondria have flat shelf-like cristae, but those in steroid secreting cells may have tubular cristae. The mitochondiral matrix contains the enzymes of the citric acid cycle, fatty acid oxidation and mitochondrial nucleic acids.

Mitochondiral DNA is double stranded and circular. Mitochndrial RNA comes in the three standard varieties; ribosomal, messenger and transfer, but each is specific to the mitochondria. Some protein synthesis occurs in the mitochondria on mitochondrial ribosomes that are different than cytoplasmic ribosomes. Other mitochondrial proteins are made on cytoplasmic ribosomes with a signal peptide that directs them to the mitochondria. The metabolic activity of the cell is related to the number of cristae and the number of mitochondria within a cell. Cells with high metabolic activity, such as heart muscle, have many well developed mitochondria. New mitochondria are formed from preexisting mitochondria when they grow and divide.

The inner membranes of mitochondria contain a family of proteins of related sequence and structure that transport various metabolites across the membrane. Their amino acid sequences have a tripartite structure, made up of three related sequences about 100 amino acids in length. The repeats of one carrier are related to those present in the others and several characteristic sequence features are conserved throughout the family.

2.1.2 Chloroplasts

In another embodiment, modified vectors disclosed herein specifically deliver polynucleotides to chloroplasts. The chloroplast is a photosynthetic organelle in eukaryotes with a double surrounding membrane. The fluid inside the double-membrane is called the stroma. The chloroplast has a nucleoid region to house its circular, naked DNA. The stroma is also the site of the Calvin Cycle. The Calvin Cycle is the series of enzyme-catalyzed chemical reactions that produce carbohydrates and other compounds from carbon dioxide.

Within the stroma are tiny membrane sacs called thylakoids. The sacs are stacked in groups. Each group is called a granum. There are many grana in each chloroplast. The thylakoid membranes are the site of photosynthetic light reactions. The thylakoids have intrinsic and extrinsic proteins, some with special prosthetic groups, allowing for electrons to be moved from protein complex to protein complex. These proteins constitute an electron transport system sometimes known as the Z-scheme.

The prosthetic group for two critical membrane proteins (P680 and P700) is a chlorophyll a pigment molecule. These chlorophyll-binding proteins give the thylakoids an intense green color. The many thylakoids in a chloroplast give the chloroplast a green color. The many chloroplasts in a leaf mesophyll cell give that cell a green color. The many mesophyll cells in a leaf give the leaf a green color. The chlorophyll molecule absorbs light energy and an electron is boosted within the electron cloud in a resonating chemical structure surrounding a magnesium ion. This excited electron is removed by the surrounding electron transport proteins in the membrane. The movement of these electrons, and accompanying protons, results ultimately in the trapping of energy in a phosphate bond in ATP.

The thylakoid is thus the location for light absorption and ATP synthesis. The stroma uses the ATP to store the trapped energy in carbon-carbon bonds of carbohydrates. Some chloroplasts show developing starch grains. These represent complex polymers of carbohydrates for long-term storage.

Given the importance of mitochondria in human disease, cell proliferation, cell death, and aging, embodiments of the present disclosure encompass the manipulation of the mitochondrial genome to supply the means by which known mitochondrial diseases (LHON, MELAS, etc.) and putative mitochondrial diseases (aging, Alzheimers, Parkinsons, Diabetes, Heart Disease) can be treated. Given the bioenergetic functions of chloroplasts, the ability to introduce exogenous genes may lead to plants with increased viability in otherwise hostile environments and increased efficiency of photosynthesis. Furthermore, the expression of exogenous genes within the chloroplasts is believed to be significantly more efficient in chloroplasts relative the expression of exogenous genes introduced into the nucleus of the cell. Thus, other embodiments are directed to the transfection of chloroplasts for more effective biosynthesis strategies for commercial compounds.

Prior to the present disclosure, no effective techniques existed to introduce exogenous nucleic acids, for example DNA, and the genes they encode into mitochondria or chloroplasts using receptor-independent methods. Phylogenetically, mitochondria and chloroplasts resemble early bacteria. One embodiment of the present disclosure is directed to a system that utilizes viral vectors, and more preferably, bacterial viruses to transfect cell organelles including chloroplasts and mitochondria. This unique molecular approach to replace, augment, or otherwise modify the chloroplast and mitochondrial genome allows, for the first time, exploration of critical questions in chloroplast and mitochondrial genetics and the development of novel therapies for mitochondrial and chloroplast related diseases.

2.2 Protein Transduction Domains

In still other embodiments, compositions for transfecting cells and organelles can be delivered from the outside of a cell or organelle to the interior of the cell or organelle by operably linking the compositions to a protein transduction domain (PTD). These small regions of proteins are able to cross the cell membrane in a receptor-independent mechanism. Although several of these PTD's have been documented, the two most commonly employed PTDs are derived from TAT (Frankel and Pabo, 1988) protein of HIV and Antennapedia transcription factor from Drosphila, whose PTD is known as Penetratin. (Derossi et al., 1994)

The Antennapedia homeodomain is 68 amino acid residues long and contains four alpha helices. Penetratin (SEQ. ID NO. 1) is an active domain of this protein which consists of a 16 amino acid sequence derived from the third helix of Antennapedia. (Fenton et al., 1998) TAT protein (SEQ. ID NO. 2) consists of 86 amino acids and is involved in the replication of HIV-1. The TAT PTD consists of an 11 amino acid sequence domain (residues 47 to 57; YGRKKRRQRRR (SEQ. ID. NO. 3)) of the parent protein that appears to be critical for uptake (Vives et al., 1997). Additionally, the basic domain Tat(49-57) or RKKRRQRRR (SEQ. ID NO. 4) (Wender et al. 2000) has been shown to be a PTD. In the current literature TAT has been favored for fusion to proteins of interest for cellular import. Several modifications to TAT, including substitutions of Glutatmine to Alanine, i.e., Q→A, have demonstrated an increase in cellular uptake anywhere from 90% (Wender et al. 2000) to up to 33 fold in mammalian cells. (Ho et al. 2001) The most efficient uptake of modified proteins was revealed by mutagenesis experiments of TAT-PTD, showing that an 11 arginine stretch was several orders of magnitude more efficient as an intercellular delivery vehicle.

2.2.1 Properties of TAT-PTD

2.2.1.1 Highly Efficient Uptake

Intracellular delivery of various therapeutic proteins involving TAT-PTD fusions have proven to be quite effective. This type of fusion protein was recently utilized in the delivery of biologically active heat shock protein 70 (HSP70) into HSF −/− cells and was compared to delivery of recombinant HSP7 for the ability to confer cytoprotection against thermal stress and hyperoxia. Immunocytochemistry demonstrated accumulation of intracellular HSP70 in nearly 100% of cells treated with the TAT-PTD fusion while the cells treated with recombinant HSP70 demonstrated no intracellular accumulation of recombinant HSP70 protein. (D. S. Wheeler et al, 2003) Other antioxidant enzymes such as superoxide dismutase (SOD) and catalase (CAT) have also been fused to TAT for intracellular delivery. With the introduction of Tat-SOD and Tat-CAT, HeLa cells experienced an approximately 90% increase in cell viability as compared to controls. (Jin et al, 2001)

Intraperitoneal (i.p.) injection of TAT-PTD anti-apoptotic protein fusions have also demonstrated significant uptake and efficacy in neuronal cells. Intraperitoneal injection of PTD-HA-Bcl-xL into mice within 1-2 hours demonstrated the ability of fusion proteins to cross the blood brain barrier. The protein fusion was able to decrease cerebral infarction up to 40% upon initiation of cerebral ischemia. (Cao, et al. 2002) A similar study utilized a Bcl-x mutant (FNK), with increased anti-apoptotic activity, to protect SH-SY5Y neurblastoma cells in vitro when exposed to staurosporine-induced apoptosis and glutamate-induced excitotoxicity. This Tat-FNK fusion was also injected i.p. into gerbils and prevented delayed neuronal death in the hippocampus caused by transient global ischemia. (Asoh, 2002)

2.2.1.2 Kinetics of Tat-PTD Fusions

Kinetic studies on the uptake of Tat-PTD have shown that an entire cell population can reach maximum uptake of the Tat-PTD within 30 seconds to 5 minutes of exposure. (Ho et al, 2001) Tat-PTD fusion proteins vary in uptake in a tissue specific manner and also depend on the structure and size of the protein fused. Stability of tranduced fusion proteins into cultured HeLa cells demonstrated a peak concentration at approximately two hours of incubation with a steady decrease up to seventy two hours later. (Jin et al 2001) Tat-PTD has also been attached to liposomes encapsulating HPTS to assess the ability of Tat-PTD to facilitate uptake of liposomal drugs into cells. Kintetic studies demonstrated accumulation in a time dependent manner. A peak concentration was achieved at approximately 24 hours and the Tat-PTD liposome fusion achieved a four fold increase in uptake as compared to Penetratin-PTD liposome fusions. (Tseng et al 2002) Tat-PTD has also been fused to Angiotensin II type I receptor (AT₁R) to investigate Tat-PTD fusion's transduction efficacy and functionality in neurons. Neuronal cultures, isolated from the hypothalamus and brainstem of 1 day old Wistar-Kyoto rats (WKY), were incubated with 300 ug/ML of the recombinant protein and peak florescence was noted at 30 minutes of incubation with initial fluorescence recorded within minutes. (Vazquez et al 2002) The figure below demonstrates that uptake of Tat-ptd fusions varies based on the type of fusion as well as cellular target for uptake.

2.2.1.3 Cytotoxicity

The parent protein of the Tat-PTD, TAT-HIV-1 protein (SEQ. ID NO. 2), elicits inflammatory responses in several cell types. Brain microvascular endothelial cells (BMEC) exposed to Tat demonstrate marked increased levels of cellular oxidative stress, decreased levels of intracellular glutathione and activated DNA binding activity and transactivation of NF-kappaB and AP-1. (Toborek et al 2003). In noting the toxicity of the parent protein the fear that Tat-PTD may exhibit similar cytotoxicity when introduced into cell culture or animal models was legitimate. However, the literature to date indicates that the Tat-PTD can transduce proteins of interest to nearly 100% of a cell population without exhibiting cytotoxic effects. To overcome the difficulty of transducing primary cultures of bone cells with proteins of interests, Tat-PTD was fused to Hemaaglutinin and calcineurin to assess transduction efficiency. Exposure to both osteoblastas and osteoclasts in primary culture led to an almost 99% tranduction of the fusion protein with retention in approximately 50% of the cell for up to five days. No cytotoxicity was reported upon treatement with the Tat-PTD fusion. (Svetlana et al 2002) Tat-PTD fusions have also been efficacious in tranducing pancreatic islets. To test whether Tat-PTD fusions were functional in islets, Tat-PTD was fused to β-galactosidase and introduced into insulinoma βTC-3 cells. Nearly 100% of all cells were transduced after a 3 hour incubation. A key requirement for any therapeutic intervention with a Tat-PTD fusion is that no untoward changes in normal cell physiology or function occur. In order to ensure proper secretion of insulin, islets were transduced with Tat-PTD-Bcl-XL. The fusion was shown to reverse hyperglycemia in diabetic nude mice with the same time frame as control islets with no concomitant toxicity, thus confirming the promising use of Tat-PTD fusion proteins as potential therapeutic interventions. (Embury, et al., 2001).

2.3 Organelle Targeting Signals: Mitochondria and Chloroplasts

In still other embodiments, targeting specific polynucleotides to organelles can be accomplished by modifying vectors to express specific organelle targeting sequences, signals, or domains. These sequences target specific organelles, but in some embodiments the interaction of the targeting sequence with the organelle does not occur through a traditional receptor:ligand interaction. The eukaryotic cell comprises a number of discrete membrane bound compartments, or organelles. The structure and function of each organelle is largely determined by its unique complement of constituent polypeptides. However, the vast majority of these polypeptides begin their synthesis in the cytoplasm. Thus organelle biogenesis and upkeep require that newly synthesized proteins can be accurately targeted to their appropriate compartment. This is often accomplished by amino-terminal signaling sequences, as well as post-translational modifications and secondary structure. For mitochondria and chloroplasts, several amino-terminal targeting sequences have been deduced and are included, in part, in Tables 1 and 2.

In one embodiment, the organelle targeting sequence can contain at least two, preferably 5-15, most preferably about 11 charged groups, causing the targeting sequence to be drawn to organelles having a net opposite charge. In another embodiment, the targeting sequence can contain a series of charged groups that cause the targeting sequence to be transported into an organelle either against or down an electromagnetic potential gradient. Suitable charged groups are groups that are charged under intracellular conditions such as amino acids with charged functional groups, amino groups, nucleic acids, and the like. Mitochondrial localization/targeting signals generally consist of a leader sequence of highly positively charged amino acids. This allows the protein to be targeted to the highly negatively charged mitochondria. Unlike receptor:ligand approaches that rely upon stochastic Brownian motion for the ligand to approach the receptor, the mitochondrial localization signal is drawn to mitochondria because of charge.

In order to enter the mitochondria, a protein generally must interact with the mitochondrial import machinery, consisting of the Tim and Tom complexes (Translocase of the Inner/Outer Mitochondrial Membrane). With regard to the mitochondrial targeting sequence, the positive charge draws the linked protein to the complexes and continues to draw the protein into the mitochondria. The Tim and Tom complexes allow the proteins to cross the membranes. Accordingly, one embodiment of the present disclosure delivers compositions of the present disclosure to the inner mitochondrial space utilizing a positively charged targeting sequence and the mitochondrial import machinery.

In yet another embodiment, the compositions of the present disclosure include an organelle targeting sequence, for example a mitochondria targeting sequence, operably linked to a protein transduction domain. The targeting sequence can be a positively charged sequence as discussed above, and typically does not operate through traditional receptor:ligand mechanisms. The PTD domain can be an HIV TAT sequence that assists the compositions in crossing lipid bilayers such as organelle membranes or plasma membranes.

2.4 Expression of Proteins on Viral Heads: Phage Display

Suitable vectors of the present disclosure include, but are not limited to, viral vectors such as bacteriophage lambda. Bacteriophage lambda has emerged as an alternative vehicle for the surface display of peptides and proteins to the commonly used filamentous phage. There are a number of unique features that make lambda an attractive display vehicle including the ability to display multimeric proteins, no requirement for secretion of the displayed fusion protein and the means to vary the valency of the displayed fusion protein. Protein D (gpD) is an established fusion partner for phage display, fused at its N- or C-terminus (Sternberg and Hoess, PNAS 92, 1609 (1995); Mikawa et al., JMB 262, 21 (1996)). Protein D is a small major capsid protein (109 aa) which contributes to the stabilization of the phage head where it forms trimeric protrusions on the phage head. Protein D is a very efficient fusion partner for high level cytoplasmic expression of soluble heterologous proteins (Forrer and Jaussi, Gene 224, 24 (1998)).

2.5 Modified Cells and Vectors

One embodiment provides a vector including a recombinant DNA sequence comprising an organelle localization signal operably linked to a sequence encoding a protein transduction domain. In this embodiment, the recombinant viral vector is engineered to contain a protein transduction domain selected for transducing the viral vector across cellular membranes. Thus, the use of such modified vectors eliminates the need for viral vectors that require transfection methods to introduce the viral vector into the cellular interior. For example, if the viral vector is a lambda bacteriophage, then the viral capsid proteins are modified to express a PTD and an organelle targeting signal on the viral surface.

In accordance with another embodiment a recombinant bacteriophage expressing a PTD and an mitochondrial targeting sequence is provided which can be used for organelle transfection, for example mitochondrial transfection, and more preferably lambda bacteriophage can be used to introduce exogenous nucleic acid sequences into mammalian mitochondria. This approach allows for direct manipulation of mtDNA and introduction of the circular genome at high-copy number, relying upon the properties of bacteriophage lambda to infect the cell's mitochondria. In particular, the 50 kilobase bacteriophage lambda genome can be engineered with large (>10 kilobase) inserts and packaged to form active lambda phage. In one embodiment, the only lambda sequences contained in the vector are the two cos sites (12 bp each) located at the 5′ and 3′ ends of a linear fragment to be packaged in a lambda particle, leaving up to about 50 kb available for a nucleic acid sequence of interest. The recombinant sequences may also include an origin of replication (usually ColE1) which allows replication in bacteria, and a gene coding for a selectable marker.

Another embodiment provides a recombinant lambda particle which displays a protein transduction domain and an organelle targeting signal. The lambda particle also includes a polynucleotide to be delivered to the organelle. Organelles can be transfected by contacting cells with the lambda particle. The lambda particle translocates across the cellular plasma membrane via the protein transduction domain and is targeted to the organelle via the targeting signal. The polynucleotide is then delivered to the organelle.

Because the minimal mitochondrial replicon is unknown, the complete human mitochondrial genome or partial fragment thereof can be inserted into lambda. In one embodiment this method is used to manipulate or replace mtDNA. In another embodiment the entire human mitochondrial genome (SEQ ID NO. 6) can be replaced by introduced sequences. For example, Rho⁰ cells can be first generated to remove endogenous mtDNA, followed by mitochondrial transfection, resulting in the entire mitochondrial genome of cells being replaced. Altematively, mitochondria can be transfected without first proceeding with the generation of Rho⁰ cells. In this case the introduced nucleic acid will be incorporated (recombined) with the existing endogenous mtDNA sequences resulting in the manipulation of the mtDNA sequences. Either method can be used to restore full functionality to damaged mitochondria.

In still another embodiment, a lambda phage vector is used wherein the structural capsid proteins of the bacteriophage have been modified to allow the bacteriophage to cross the cellular membrane using a PTD and targeted to the mitochondria using a mitochondrial targeting signal. Preferably the modified phage vector protein is one that allows for the expression of protein moieties such as PTD and an organelle targeting signal. Wild type lambda phage expresses gpD on its capsid or head. The viral capsid is composed of several proteins, including the gpD protein, and this protein can be modified to produce a bacteriophage that is capable of specifically crossing cellular membranes and targeting to organelles.

Suitable mitochondria localization sequences are known to those skilled in the art (see Table 1) and include the mitochondrial localization signal of subunit VIII of human cytochrome oxidase, the yeast cytochrome c oxidase subunit IV presequence and the amino-terminal leader peptide of the rat ornithine-transcarbamylase. In one embodiment the introduced sequences are expressed on the viral capsid head. Upon expression of the recombinant viral vector, the mitochondrial localization signal causes the viral vector to be localized to the mitochondria. Transfection of this host cell with a suitable viral vector will target the vector to the mitochondria.

Nucleic acids encoding a protein of a vector, for example a viral capsid protein for a viral vector, can be operatively linked to an organelle targeting sequence, for example amino acids used to import proteins into the mitochondria. This hybrid protein can be used to target nucleic acids to the organelle. Once inside the organelle, the nucleic acid can be integrated into the genome of the organelle. Thus, an embodiment of the present disclosure is directed to a polypeptide comprising at least a partial viral capsid protein sequence and organelle targeting sequence, for example the targeting sequences of Table 1 or Table 2. The hybrid polypeptide can be expressed independently or can be expressed as part of a viral vector.

Another embodiment provides a transfection system that comprises two components, the viral vector modified to express a PTD that delivers the nucleic acid of interest, for example RNA, DNA, or a combination thereof, to the cellular interior and a organelle targeting signal, wherein the construct comprises a nucleic acid sequence that encodes an organelle localization sequence operably linked to a viral surface protein specific for the viral vector. The PTD and the organelle targeting sequence can be expressed as separate polypeptides on the surface of the viral vector or in a single polypeptide sequence, fusion protein, on the surface of the viral vector. The nucleic acid construct also optionally includes a suitable promoter for expressing the fusion protein as well as any other necessary regulatory elements for expressing the fusion protein. Such regulatory elements are well known to those skilled in the art and will vary based on the fusion protein expression system. One viral vector suitable for use with the present disclosure is bacteriophage lambda. When bacteriophage lambda is used as the transfection vector, the component of the transfection system comprises a nucleic acid sequence encoding the lambda capsid protein(gpD) operably linked to the organelle localization sequence and protein transduction domain.

Recombinant viral vectors that comprise modified organelle targeting sequences that are expressed on the viral surface can be prepared using standard molecular biology techniques. In general, a host cell is transfected with a recombinant viral construct comprising a sequence that encodes an organelle localization signal and protein transduction domain operably linked to a sequence encoding the viral surface protein, for example a viral capsid protein. The organelle localization sequence allows a protein that is linked to the localization sequence (i.e., a fusion protein) to be delivered to the target organelle. According to one embodiment of the present disclosure, the localization sequence is used to target a viral vector to an organelle of choice, for example mitochondria or chloroplast, and thus provide a point for the introduced vector that targets to the organelle. The vector is introduced into the cytosol of the cell through its protein transduction domain and then binds to the organelle specific for the vector. The nucleic acid of interest within the vector is delivered into the target animal or plant organelle.

Organelle localization signals are known to those skilled in the art, and any of those signals can be used to target the viral vector to the target organelle. Localization sequences suitable for use in the present disclosure are described in Emahuelson et al., Predicting Subcellular Localization of Proteins Based on Their N-terminal Amino Acid Sequence. Journal of Molecular Biology. 300(4):1005-16, 2000 Jul. 21, and in Cline and Henry, Import and Routing of Nucleus-encoded Chloroplast Proteins. Annual Review of Cell & Developmental Biology. 12:1-26, 1996, the disclosures of which are incorporated herein by reference in their entirety. More particularly, a list of mitochondria localization signals for targeting linked proteins or nucleic acids to the mitochondria is listed in TABLE 1. A list of chloroplast localization signals for targeting linked proteins or nucleic acids to the chloroplasts is listed in TABLE 2. In one embodiment the mitochondria or chloroplast localization signal is operably linked to a virus surface protein. It will be appreciated that part or all of the sequences listed in Tables 1 and 2 can be used as organelle targeting sequences.

TABLE 1
Localization Signals for Targeting to the Mitochondria.
(verified using Mitochondrial Project MITOP Database -
http://mips.gsf.de/proj/medgen/mitop/)

| MITOP | SEQ. ID. | Gene | |
|---|---|---|---|
| Designation | NO. | Name | Gene Name Full |
| 106092 | 7 | Etfa | electron transfer flavoprotein alpha chain |
| | | | precursor - mouse (SEQ ID NO. 7) |
| 106098 | 8 | Etfb | electron transfer flavoprotein beta chain - |
| | | | mouse (SEQ ID NO. 8) |
| 107450 | 9 | Dld | dihydrolipoamide dehydrogenase precursor - |
| | | | human (SEQ ID NO. 9) |
| 87979 | 10 | Ak3 | nucleoside-triphosphate- adenylate kinase 3 - |
| | | | mouse (SEQ ID NO. 10) |
| 88529 | 11 | Cs | citrate synthase, mitochondrial (SEQ ID NO. |
| | | | 11) |
| 891996 | 12 | Cps1 | carbamoyl-phosphate synthetase 1 (SEQ ID |
| | | | NO. 12) |
| 97045 | 13 | Mod2 | malic enzyme complex, mitochondrial - mouse |
| | | | (SEQ ID NO. 13) |
| 97499 | 14 | Pcca | propionyl-CoA carboxylase alpha chain |
| | | | precursor - mouse (SEQ ID NO. 14) |
| A27883 | 15 | PCCA | propionyl-CoA carboxylase alpha chain |
| | | | precursor (SEQ ID NO. 15) |
| A28053 | 16 | Cbr2 | carbonyl reductase (NADPH) - mouse (SEQ |
| | | | ID NO. 16) |
| A29881 | 17 | mpp-2 | Mitochondrial processing peptidase beta |
| | | | subunit precursor (beta-mpp) (ubiquinol- |
| | | | cytochrome c reductase complexcore protein I) |
| | | | (SEQ ID NO. 17) |
| A30605 | 18 | ACADS | acyl-CoA dehydrogenase precursor, short- |
| | | | chain-specific (SEQ ID NO. 18) |
| A31998 | 19 | ETFA | electron transfer flavoprotein alpha chain |
| | | | precursor (SEQ ID NO. 19) |
| A32422 | 20 | DBT | Dihydrolipoamide S-(2- |
| | | | methylpropanoyl)transferase precursor (SEQ ID |
| | | | NO. 20) |
| A32800 | 21 | HSPD1 | heat shock protein 60 precursor (SEQ ID NO. |
| | | | 21) |
| A36442 | 22 | mpp-1 | Mitochondrial processing peptidase alpha chai [symbol] |
| | | | precursor (SEQ ID NO. 22) |
| A37033 | 23 | IVD | isovaleryl-CoA dehydrogenase precursor (SE [symbol] |
| | | | ID NO. 23) |
| A37157 | 24 | BCKD | 3-methyl-2-oxobutanoate dehydrogenase |
| | | | (lipoamide) E1-beta chain precursor |
| | | | (SEQ ID NO. 24) |
| A38234 | 25 | OGDH | Oxoglutarate dehydrogenase (lipoamide) |
| | | | precursor (SEQ ID NO. 25) |
| A39503 | 26 | ME2 | Malate dehydrogenase (NAD+) precursor, |
| | | | mitochondrial (SEQ ID NO. 26) |
| A40487 | 27 | FDXR | ferredoxin-NADP+ reductase, long form, |
| | | | precursor (SEQ ID NO. 27) |
| A40559 | 28 | ACADL | long-chain-acyl-CoA dehydrogenase (LCAD) |
| | | | (SEQ ID NO. 28) |
| A40872 | 29 | ALDH5 | aldehyde dehydrogenase (NAD+) 5 precursor, |
| | | | mitochondrial (SEQ ID NO. 29) |
| A41581 | 30 | CYP3 | peptidylprolyl isomerase 3 precursor |
| | | | (SEQ ID NO. 30) |
| A42224 | 31 | arg-2 | Carbamoyl-phosphate synthase, arginine- |
| | | | specific, small chain precursor (arginine-specif [symbol] |
| | | | carbamoyl-phosphate synthetase, glutamine |
| | | | chain) (cps-a) |
| | | | (SEQ ID NO. 31) |
| A42845 | 32 | BDH | D-beta-hydroxybutyrate dehydrogenase |
| | | | precursor (3-hydroxybutyrate dehydrogenase) |
| | | | (fragment) (SEQ ID NO. 32) |
| A45470 | 33 | HMGC | Hydroxymethylglutaryl-CoA lyase |
| | | | (SEQ ID NO. 33) |
| A47255 | 34 | Pcx | pyruvate carboxylase (SEQ ID NO. 34) |
| A53020 | 35 | PCCB | propionyl-CoA carboxylase beta chain |
| | | | precursor (SEQ ID NO. 35) |
| A53719 | 36 | GLUDP | glutamate dehydrogenase (NAD(P)+) 2 |
| | | | precursor (SEQ ID NO. 36) |
| A55075 | 37 | HspE1 | chaperonin-10 (SEQ ID NO. 37) |
| A55680 | 38 | ACADS | short/branched chain acyl-CoA dehydrogenase |
| | | | precursor (SEQ ID NO. 38) |
| A55723 | 39 | DCI | dodecenoyl-CoA Delta-isomerase precursor, |
| | | | mitochondrial (SEQ ID NO. 39) |
| A55724 | 40 | Acadm | Acyl-CoA dehydrogenase, medium-chain |
| | | | specific precursor (MCAD) (SEQ ID NO. 40) |
| AA227572 | 41 | WARS2 | tryptophanyl-tRNA synthetase 2 (mitochondrial - |
| | | | human (SEQ ID NO. 41) |
| AB029948 | 42 | SerRS | mitochondrial seryl-tRNA synthetase (cDNA |
| | | | FLJ20450 FIS, CLONE KAT05607) - human |
| | | | (SEQ ID NO. 42) |
| ACDL_MOUSE | 43 | Acadl | Acyl-CoA dehydrogenase, long-chain specific |
| | | | precursor (LCAD) (SEQ ID NO. 43) |
| AF047042 | 44 | CS | citrate synthase, mitochondrial (SEQ ID NO. |
| | | | 44) |
| AF097441 | 45 | FARS1 | phenylalanine-tRNA synthetase (FARS1) |
| | | | mRNA, nuclear gene encoding mitochondrial |
| | | | protein - human (SEQ ID NO. 45) |
| ATPO_HUMAN | 46 | ATP5O | ATP synthase oligomycin sensitivity conferral |
| | | | protein precursor, mitochondrial (SEQ ID NO. |
| | | | 46) |
| AXHU | 47 | FDX1 | adrenodoxin precursor (SEQ ID NO. 47) |
| CCHU | 48 | HCS | cytochrome c (SEQ ID NO. 48) |
| CCNC | 49 | cyc-1 | Cytochrome c (SEQ ID NO. 49) |
| CE06620 | 50 | — | Probable leucyl-tRNA synthetase, mitochondri [symbol] |
| | | | (SEQ ID NO. 50) |
| CE09597 | 51 | — | Pyruvate dehydrogenase (E2) dihydrolipoamid |
| | | | acetyltransferase (SEQ ID NO. 51) |
| CH10_MOUSE | 52 | Hspe1 | 10 KD heat shock protein, mitochondrial |
| | | | (hsp10) (10K chaperonin) mouse (SEQ ID NO. |
| | | | 52) |
| CH60_CAEEL | 53 | hsp60 | Chaperonin homolog hsp60 precursor (heat |
| | | | shock protein 60) (hsp-60) (SEQ ID NO. 53) |
| DEHUE2 | 54 | ALDH2 | aldehyde dehydrogenase (NAD+) 2 precursor, |
| | | | mitochondrial (SEQ ID NO. 54) |
| DEHUE | 55 | GLUD1 | glutamate dehydrogenase (NAD(P)+) precurso |
| | | | (SEQ ID NO. 55) |
| DEHULP | 56 | DLD | Dihydrolipoamide dehydrogenase precursor |
| | | | (SEQ ID NO. 56) |
| DEHUPA | 57 | PDHA1 | pyruvate dehydrogenase (lipoamide) alpha |
| | | | chain precursor (SEQ ID NO. 57) |
| DEHUPB | 58 | PDHB | pyruvate dehydrogenase (lipoamide) beta chai |
| | | | precursor (SEQ ID NO. 58) |
| DEHUPT | 59 | PDHA2 | pyruvate dehydrogenase (lipoamide) alpha |
| | | | chain precursor, testis-specific (E1) (SEQ ID |
| | | | NO. 59) |
| DEHUXA | 60 | BCKDH | 3-methyl-2-oxobutanoate dehydrogenase |
| | | | (lipoamide) alpha chain precursor (SEQ ID NO |
| | | | 60) |
| DEMSMM | 61 | Mor1 | malate dehydrogenase precursor, mitochondri [symbol] |
| | | | (SEQ ID NO. 61) |
| DSHUN | 62 | SOD2 | superoxide dismutase (Mn) precursor |
| | | | (SEQ ID NO. 62) |
| ECHM_HUMAN | 63 | ECHS1 | enoyl-CoA hydratase, mitochondrial (short |
| | | | chain enoyl-CoA hydratase (SCEH) (SEQ ID |
| | | | NO. 63) |
| GABT_HUMAN | 64 | ABAT | 4-aminobutyrate aminotransferase, |
| | | | mitochondrial precursor (gamma-amino-N- |
| | | | butyrate-transaminase) (GABA transaminase) |
| | | | (SEQ ID NO. 64) |
| GCDH_HUMAN | 65 | GCDH | glutaryl-CoA dehydrogenase precursor (GCD) |
| | | | human (SEQ ID NO. 65) |
| GCDH_MOUSE | 66 | Gcdh | Glutaryl-CoA dehydrogenase precursor (GCD) - |
| | | | mouse (SEQ ID NO. 66) |
| HCD1_CAEEL | 67 | — | Probable 3-hydroxyacyl-CoA dehydrogenase |
| | | | F54C8.1 (SEQ ID NO. 67) |
| HCD2_CAEEL | 68 | — | Probable 3-hydroxyacyl-CoA dehydrogenase |
| | | | B0272.3 (SEQ ID NO. 68) |
| HHMS60 | 69 | Hsp60 | heat shock protein 60 precursor (SEQ ID NO. |
| | | | 69) |
| HMGL_MOUSE | 70 | Hmgcl | hydroxymethylglutaryl-CoA lyase precursor |
| | | | (HG-CoA lyase) (HL) (3-hydroxy-3- |
| | | | methylglutarate-CoA lyase) (SEQ ID NO. 70) |
| I48884 | 71 | — | 2-oxoglutarate dehydrogenase E1 component |
| | | | (fragment) (SEQ ID NO. 71) |
| I48966 | 72 | Aldh2 | aldehyde dehydrogenase (NAD+) 2 precursor, |
| | | | mitochondrial (SEQ ID NO. 72) |
| I49605 | 73 | Acads | Acyl-CoA dehydrogenase, short-chain specific |
| | | | precursor (SCAD) (butyryl-CoA |
| | | | dehydrogenase) (SEQ ID NO. 73) |
| I52240 | 74 | ACAD | acyl-CoA dehydrogenase precurser, medium- |
| | | | chain-specific (SEQ ID NO. 74) |
| I55465 | 75 | PDK1 | pyruvate dehydrogenase kinase isoform 1 - |
| | | | human (SEQ ID NO. 75) |
| I57023 | 76 | Sod2 | superoxide dismutase (Mn) precursor |
| | | | (SEQ ID NO. 76) |
| I70159 | 77 | PDK2 | Pyruvate dehydrogenase kinase isoform 2 - |
| | | | human (SEQ ID NO. 77) |
| I70160 | 78 | PDK3 | pyruvate dehydrogenase kinase isoform 3 - |
| | | | human (SEQ ID NO. 78) |
| JC2108 | 79 | HADH | long-chain-fatty-acid beta-oxidation |
| | | | multienzyme complex alpha chain precursor, |
| | | | mitochondrial |
| | | | (SEQ ID NO. 79) |
| JC2109 | 80 | HADH | long-chain-fatty-acid beta-oxidation |
| | | | multienzyme complex beta chain precursor, |
| | | | mitochondrial |
| | | | (SEQ ID NO. 80) |
| JC2460 | 81 | PC | pyruvate carboxylase precursor (SEQ ID NO. |
| | | | 81) |
| JC4879 | 82 | SCHAD | 3-hydroxyacyl-CoA dehydrogenase, short |
| | | | chain-specific, precursor (SEQ ID NO. 82) |
| KIHUA3 | 83 | AK3 | nucleoside-triphosphate- adenylate kinase 3 |
| | | | (SEQ ID NO. 83) |
| M2GD_HUMAN | 84 | DMGD | Dimethylglycine dehydrogenase, mitochondrial |
| | | | precursor (ME2GLYDH) - human (SEQ ID NO |
| | | | 84) |
| MDHM_HUMA | 85 | MDH2 | malate dehydrogenase mitochondrial precurso |
| | | | (fragment) (SEQ ID NO. 85) |
| O75439 | 86 | PMPC | mitochondrial processing peptidase beta |
| | | | subunit precursor (beta-MPP) (P-52) (SEQ ID |
| | | | NO. 86) |
| ODO1_MOUSE | 87 | Ogdh | 2-oxoglutarate dehydrogenase E1 component |
| | | | (alpha-ketoglutarate dehydrogenase) |
| | | | (fragment) |
| | | | (SEQ ID NO. 87) |
| ODPA_CAEEL | 88 | — | Probable pyruvate dehydrogenase E1 |
| | | | component, alpha subunit precursor (PDHE1- [symbol] |
| | | | (SEQ ID NO. 88) |
| OWHU | 89 | OTC | ornithine carbamoyltransferase precursor |
| | | | (SEQ ID NO. 89) |
| OWMS | 90 | Otc | ornithine carbamoyltransferase precursor |
| | | | (SEQ ID NO. 90) |
| P21549 | 91 | AGXT | alanine- glyoxylate aminotransferase |
| | | | (SEQ ID NO. 91) |
| PUT2_HUMAN | 92 | ALDH4 | Delta-1-pyrroline-5-carboxylate dehydrogenas [symbol] |
| | | | precursor (P5C dehydrogenase) (SEQ ID NO. |
| | | | 92) |
| Q0140 | 93 | VAR1 | VAR1 - mitochondrial ribosomal protein |
| | | | (SEQ ID NO. 93) |
| Q10713 | 94 | KIAA0123 | mitochondrial processing peptidase alpha |
| | | | subunit precursor (alpha-MPP) (P-55) (HA152 [symbol] |
| | | | (SEQ ID NO. 94) |
| Q16654 | 95 | PDK4 | pyruvate dehydrogenase kinase isoform 4 - |
| | | | human |
| | | | (SEQ ID NO. 95) |
| ROHU | 96 | TST | thiosulfate sulfurtransferase (SEQ ID NO. 96) |
| S01174 | 97 | Got2 | aspartate transaminase precursor, |
| | | | mitochondrial |
| | | | (SEQ ID NO. 97) |
| S08680 | 98 | Mut | methylmalonyl-CoA mutase alpha chain |
| | | | precursor |
| | | | (SEQ ID NO. 98) |
| S13025 | 99 | nuo-40 | NADH dehydrogenase (ubiquinone) 40K chain |
| | | | (SEQ ID NO. 99) |
| S13048 | 100 | cyt | cytochrome c (SEQ ID NO. 100) |
| S16239 | 101 | Glud | glutamate dehydrogenase (NAD(P)+) precurso |
| | | | (SEQ ID NO. 101) |
| S23506 | 102 | Pdha1 | pyruvate dehydrogenase (lipoamide) |
| | | | (SEQ ID NO. 102) |
| S25665 | 103 | DLAT_h | dihydrolipoamide S-acetyltransferase heart - |
| | | | human (fragment) (SEQ ID NO. 103) |
| S26984 | 104 | — | probable DNA-directed RNA polymerase - |
| | | | mitochondrion plasmid maranhar (SGC3) |
| | | | (SEQ ID NO. 104) |
| S32482 | 105 | ETFB | electron transfer flavoprotein beta chain |
| | | | (SEQ ID NO. 105) |
| S38770 | 106 | Dci | 3,2-trans-enoyl-CoA isomerase, mitochondrial |
| | | | precursor (dodecenoyl-CoA delta-isomerase) |
| | | | (SEQ ID NO. 106) |
| S39807 | 107 | Bckdhb | 3-methyl-2-oxobutanoate dehydrogenase |
| | | | (lipoamide) beta chain (SEQ ID NO. 107) |
| S40622 | 108 | MUT | methylmalonyl-CoA mutase precursror (MCM) |
| | | | (SEQ ID NO. 108) |
| S41006 | 109 | — | hypothetical protein t05g5.6 (SEQ ID NO. 109) |
| S41563 | 110 | cit-1 | citrate (si)-synthase, mitochondrial |
| | | | (SEQ ID NO. 110) |
| S42366 | 111 | PRSS15 | Lon proteinase homolog (SEQ ID NO. 111) |
| S42370 | 112 | — | citrate synthase homolog (SEQ ID NO. 112) |
| S47532 | 113 | HSPE1 | heat shock protein 10 (SEQ ID NO. 113) |
| S53351 | 114 | ME2.1 | malate dehydrogenase (oxaloacetate- |
| | | | decarboxylating) (NADP+) precursor, |
| | | | mitochondrial |
| | | | (SEQ ID NO. 114) |
| S60028 | 115 | Fdxr | ferredoxin-NADP+ reductase precursor |
| | | | (SEQ ID NO. 115) |
| S65760 | 116 | Dbt | dihydrolipoamide transacylase precursor |
| | | | (SEQ ID NO. 116) |
| S71881 | 117 | Bckdha | branched chain alpha-ketoacid dehydrogenase |
| | | | chain E1-alpha precursor (SEQ ID NO. 117) |
| SCOT_HUMA | 118 | OXCT | Succinyl-CoA: 3-ketoacid-coenzyme A |
| | | | transferase precursor (succinyl CoA: 3-oxoacid |
| | | | CoA-transferase) (OXCT) (SEQ ID NO. 118) |
| SODM_CAEEL | 119 | sod-2 | Superoxide dismutase precursor (Mn) |
| | | | (SEQ ID NO. 119) |
| SODN_CAEEL | 120 | sod-3 | Superoxide dismutase precursor (Mn) |
| | | | (SEQ ID NO. 120) |
| SYHUAE | 121 | ALAS2 | 5-aminolevulinate synthase 2 (SEQ ID NO. |
| | | | 121) |
| SYHUAL | 122 | ALAS1 | 5-aminolevulinate synthase 1 precursor |
| | | | (SEQ ID NO. 122) |
| SYLM_HUMAN | 123 | KIAA0028 | Probable leucyl-TrNA synthetase, mitochondria |
| | | | precursor (Leucine-tRNA ligase) (Leurs) |
| | | | (KIAA0028) (SEQ ID NO. 123) |
| SYMSAL | 124 | Alas2 | 5-aminolevulinate synthase mitochondrial |
| | | | precursor (erythroid-specific) (ALAS-E) (SEQ ID |
| | | | NO. 124) |
| SYNCLM | 125 | leu-5 | leucine-tRNA ligase precursor, mitochondrial |
| | | | (SEQ ID NO. 125) |
| SYNCYT | 126 | cyt-18 | tyrosine-tRNA ligase precursor, mitochondrial |
| | | | (SEQ ID NO. 126) |
| SYWM_CAEEL | 127 | — | Probable tryptophanyl-tRNA synthetase, |
| | | | mitochondrial (tryptophan-tRNA ligase) |
| | | | (TRPRS) |
| | | | (SEQ ID NO. 127) |
| THTR_MOUSE | 128 | Tst | thiosulfate sulfurtransferase (SEQ ID NO. 128) |
| U80034 | 129 | MIPEP | mitochondrial intermediate peptidase |
| | | | (SEQ ID NO. 129) |
| U82328 | 130 | PDX1 | pyruvate dehydrogenase complex protein X |
| | | | subunit precursor (SEQ ID NO. 130) |
| XNHUDM | 131 | GOT2 | aspartate transaminase precursor, |
| | | | mitochondrial (SEQ ID NO. 131) |
| XNHUO | 132 | OAT | ornithine-oxo-acid transaminase precursor |
| | | | (SEQ ID NO. 132) |
| XNHUSP | 133 | AGXT | serine-pyruvate aminotransferase (SPT) |
| | | | (alanine-glyoxylate aminotransferase) (AGT) |
| | | | (SEQ ID NO. 133) |
| XNMSO | 134 | Oat | ornithine-oxo-acid transaminase precursor |
| | | | (SEQ ID NO. 134) |
| XXHU | 135 | DLAT | dihydrolipoamide S-acetyltransferase precurso |
| | | | (fragment) (SEQ ID NO. 135) |
| YAL044c | 136 | GCV3 | GCV3 - glycine decarboxylase, subunit H |
| | | | (SEQ ID NO. 136) |
| YBL022c | 137 | PIM1 | PIM1 - ATP-dependent protease, mitochondria |
| | | | (SEQ ID NO. 137) |
| YBL038w | 138 | MRPL16 | MRPL16 - ribosomal protein of the large |
| | | | subunit, mitochondrial (SEQ ID NO. 138) |
| YBL080c | 139 | PET112 | PET112 - required to maintain rho+ |
| | | | mitochondrial DNA (SEQ ID NO. 139) |
| YBL090w | 140 | MRP21 | MRP21 - Mitochondrial ribosomal protein |
| | | | (SEQ ID NO. 140) |
| YBR120c | 141 | CBP6 | CBP6 - apo-cytochrome B pre-mRNA |
| | | | processing protein (SEQ ID NO. 141) |
| YBR122c | 142 | MRPL36 | MRPL36 - ribosomal protein YmL36 precursor, |
| | | | mitochondrial (SEQ ID NO. 142) |
| YBR146w | 143 | MRPS9 | MRPS9 - ribosomal protein S9 precursor, |
| | | | mitochondrial (SEQ ID NO. 143) |
| YBR221c | 144 | PDB1 | PDB1 - pyruvate dehydrogenase (lipoamide) |
| | | | beta chain precursor |
| YBR227c | 145 | MCX1 | MCX1 - ClpX homologue in mitochondria |
| YBR251w | 146 | MRPS5 | MRPS5 - ribosomal protein S5, mitochondrial |
| YBR268w | 147 | MRPL37 | MRPL37 - ribosomal protein YmL37, |
| | | | mitochondrial |
| YBR282w | 148 | MRPL27 | MRPL27 - ribosomal protein YmL27 precursor, |
| | | | mitochondrial |
| YCR003w | 149 | MRPL32 | MRPL32 - ribosomal protein YmL32, |
| | | | mitochondrial |
| YCR024c | 150 | — | asn-tRNA synthetase, mitochondrial |
| YCR028c-a | 151 | RIM1 | RIM1 - ssDNA-binding protein, mitochondrial |
| YCR046c | 152 | IMG1 | IMG1 - ribosomal protein, mitochondrial |
| YDL202w | 153 | MRPL11 | MRPL11 - ribosomal protein of the large |
| | | | subunit, mitochondrial |
| YDR148c | 154 | KGD2 | KGD2 - 2-oxoglutarate dehydrogenase comple |
| | | | E2 component |
| YDR194c | 155 | MSS116 | MSS116 - RNA helicase of the DEAD box |
| | | | family, mitochondrial |
| YDR462w | 156 | MRPL28 | MRPL28 - ribosomal protein of the large subun |
| | | | (YmL28), mitochondrial |
| YFL018c | 157 | LPD1 | LPD1 - dihydrolipoamide dehydrogenase |
| | | | precursor |
| YGR244c | 158 | LSC2 | succinate-CoA ligase beta subunit |
| YHR008c | 159 | SOD2 | SOD2 - superoxide dismutase (Mn) precursor, |
| | | | mitochondrial |
| YIL070c | 160 | MAM33 | MAM33 - mitochondrial acidic matrix protein |
| YJL096w | 161 | MRPL49 | MRPL49 - ribosomal protein YmL49, |
| | | | mitochondrial |
| YJR113c | 162 | RSM7 | RSM7 - similarity to bacterial, chloroplast and |
| | | | mitochondrial ribosomal protein S7 |
| YKL040c | 163 | NFU1 | NFU1 - iron homeostasis |
| YLL027w | 164 | ISA1 | ISA1 - mitochondrial protein required for norma |
| | | | iron metabolism |
| YLR059c | 165 | REX2 | REX2 - putative 3′-5′ exonuclease |
| YML110c | 166 | COQ5 | COQ5 - ubiquinone biosynthesis, |
| | | | methyltransferase |
| YMR062c | 167 | ECM40 | ECM40 - acetylornithine acetyltransferase |
| YMR072w | 168 | ABF2 | ABF2 - high mobility group protein |
| YOL095c | 169 | HMI1 | HMI1 - mitochondrial DNA helicase |
| YOR040w | 170 | GLO4 | GLO4 - glyoxalase II (hydroxyacylglutathione |
| | | | hydrolase) |
| YOR142w | 171 | LSC1 | LSC1 - succinate-CoA ligase alpha subunit |
| YPL118w | 172 | MRP51 | MRP51 - strong similarity to S. kluyveri |
| | | | hypothetical protein |
| YPL135w | 173 | ISU1 | ISU1 - protein with similarity to iron-sulfur |
| | | | cluster nitrogen fixation proteins |
| YPL252c | 174 | YAH1 | YAH1 - similarity to adrenodoxin and ferrodoxi [symbol] |
| YPL262w | 175 | FUM1 | FUM1 - fumarate hydratase |
| YPR047w | 176 | MSF1 | MSF1 - phenylalanine-tRNA ligase alpha |
| | | | chain, mitochondrial |
| YPR067w | 177 | ISA2 | ISA2 - mitochondrial protein required for iron |
| | | | metabolism |

TABLE 2
Localization Signals for Targeting to the Chloroplast:

| | SEQ. | |
|---|---|---|
| Designation | ID NO. | Description |
| CA782533 | 178 | Transit peptide domain of the apicoblast |
| | | ribosomal protein S9 |
| P27456 | 179 | Pea glutathione reductase (GR) signal peptide |
| BAB91333 | 180 | NH₂-terminus of Cr-RSH encoding a putative |
| | | guanosine 3′,5′-bispyrophosphate (ppGpp) |
| | | synthase-degradase |
| CAB42546 | 181 | 14-3-3 proteins |
| AAC64139 | 182 | Chloroplast signal recognition particle |
| AAC64109 | 183 | including cpSRP54, cpSRP43 subunits |
| AAD01509 | 184 | or a fragment thereof |
| PWSPG, | 185 | Chloroplast transit peptides |
| FESP1, | 186 | |
| P00221, | 187 | |
| P05435, | 188 | |
| BAA37170, | 189 | |
| BAA37171, | 190 | |
| AAA81472 | 191 | |
| X52428 | 192 | AtOEP7, in particular the transmembrane |
| | | domain (TMD) and its C-terminal neighboring |
| | | seven-amino acid region (see Lee YJ, |
| | | Plant Cell 2001 Oct; 13(10): 2175-90) |
| CA757092, | 193 | THI1 N-terminal chloroplastic transit peptide, |
| CA755666 | 194 | in particular 4 to 27 residues |

The identification of the specific sequences necessary for translocation of a linked protein into a chloroplast or mitochondria can be determined using predictive software known to those skilled in the art, including the tools located at http://www.mips.biochem.mpg.de/cgi-bin/proj/medgen/mitofilter.

In another embodiment, a nucleic acid encoding a vector containing a protein transduction domain and organelle localization signal can be introduced into organelles of cells from a host, primary culture, or a cell line. For example, a viral vector operatively linked with an organelle targeting sequence can be used to transfect a eukaryotic cell line such that the nucleic acid sequence is stably integrated into the organelle genome of a cell of the cell line. The cell line can be a transformed cell line that can be maintained indefinitely in cell culture, or the cell line can be a primary cell culture. Exemplary cell lines are those available from American Type Culture Collection including plant cell lines which are incorporated herein by reference. The nucleic acid can be replicated and transcribed within the nucleus of a cell of the transfected cell line. The targeting sequence can be enzymatically cleaved if necessary such that the vector is free to remain in the target organelle.

Any eukaryotic cell can be transfected to produce organelles that express a specific nucleic acid, for example a metabolic gene, including primary cells as well as established cell lines. Suitable types of cells include but are not limited to undifferentiated or partially differentiated cells including stem cells, totipotent cells, pluripotent cells, embryonic stem cells, inner mass cells, adult stem cells, bone marrow cells, cells from umbilical cord blood, and cells derived from ectoderm, mesoderm, or endoderm. Suitable differentiated cells include somatic cells, neuronal cells, skeletal muscle, smooth muscle, pancreatic cells, liver cells, and cardiac cells. Suitable plant cells can be selected from monocots and dicots, and include corn, soybeans, legumes, grasses, and grains such as rice and wheat.

If the organelle to be targeted is a chloroplast, then the host cell can be selected from known eukaryotic photosynthetic cells. If the organelle to be transfected is the mitochondrion, than any eukaryotic cell can be used, including mammalian cells, for example human cells. The cells are transfected to either transiently or stably express the exogenous nucleic acid. In one embodiment a DNA construct encoding a reporter gene is integrated into the mitochondrial genome of a cell to produce a stable transgenic cell line that comprises organelles that express the desired reporter gene.

In another embodiment, siRNA or antisense polynucleotides (including siRNA or antisense polynucleotides directed to mtDNA related proteins) can be transfected into an organelle using the compositions described herein.

Another embodiment of the disclosure provides a cell having a modified organelle, wherein the modified organelle includes an exogenously introduced nucleic acid. An exogenous nucleic acid means a nucleic acid not naturally associated with the organelle or located in the organelle's interior. The nucleic acid expressed in the organelle can be transcribed and/or translated within the organelle. Additionally, the nucleic acid and its resultant protein can undergo posttranslational modification within the organelle, if necessary, to facilitate its function. Delivery of modified viral vectors to specific organelles can be accomplished using targeting sequences, for example the targeting sequences in Table 1.

Nucleic acids including but not limited to polynucleotides, anti-sense nucleic acids, peptide nucleic acids, natural or synthetic nucleic acids, nucleic acids with chemically modified bases, RNA, DNA, RNA-DNA hybrids, enzymatic nucleic acids such as ribozymes and DNAzymes, native/endogenous genes and non-native/exogenous genes and fragments or combinations thereof, can be introduced into organelles of a host cell, in particular organelles that can transcribe and or translate nucleic acids into proteins such as mitochondria and chloroplasts. In one embodiment of the present disclosure, all or part of the mitochondrial or chloroplastic genome can be introduced into an organelle. The nucleic acids can be introduced into the organelle with the vector when the vector crosses the organelle membrane via protein transduction domains.

3 Methods

In accordance with the present disclosure one exemplary method for transfecting a cellular organelle, for example non-nuclear organelles such as the mitochondria and chloroplasts, comprises the steps of contacting a cell with a recombinant vector, for example a viral vector, wherein the vector includes a protein transduction domain and an organelle targeting signal located on the surface of the vector. Suitable cells include cells capable of being transfected, for example eukaryotic cells. Organelle targeting signals of the present disclosure include polypeptides having a net positive charge and those listed in Tables 1 and 2. Suitable PTDs include but are not limited to HIV TAT YGRKKRRQRRR (SEQ. ID NO. 3) or RKKRRQRRR (SEQ. ID NO. 4); 11 Arginine residues, or positively charged polypeptides or polynucleotides having 8-15 residues, preferably 9-11 residues. The term non-nuclear organelle is intended to encompass all organelles other than the nucleus. It will be appreciated the viral vector express a organelle targeting signal which causes the vector to associate with the organelle, typically to an organelle having a net negative charge or a region having a negative charge. In one embodiment, the association of the targeting signal with the organelle does not occur through a receptor:ligand interaction. The association of the organelle and vector can be ionic, non-covalent, covalent, reversible or irreversible. Exemplary vector:organelle associations include but are not limited to protein-protein, protein-carbohydrate, protein-nucleic acid, nucleic acid-nucleic acid, protein-lipid, lipid-carbohydrate, antibody-antigen, or avidin-biotin. The organelle targeting signal on the surface of the vector can be a protein, peptide, antibody, antibody fragment, lipid, carbohydrate, biotin, avidin, steptavidin, chemical group, or other ligand that causes specific association between the organelle and vector, preferably an electromagnetic association as between oppositely charged moieties.

The specific interaction between the introduced vector and its target organelle can be accomplished by at least two methods. In one exemplary method a recombinant viral vector can be genetically engineered to express a targeting signal, for example as a component of a polypeptide expressed on the exterior of the vector so that the targeting signal is free to interact with the targeted the organelle. Preferably, the vector expresses a surface polypeptide that is specific to the target organelle. In another method the vector is modified to incorporate an exogenous targeting protein to which an organelle binds. Alternatively, a vector can be modified to specifically interact with a desired organelle, for example by expressing an antibody fragment that can bind to an epitope on a specific organelle. It will be appreciated by those of skill in the art that the vector can be chemically modified to have a net positive or negative charge depending on the modification agent. For example, a vector can be coated with polylysine or other agents containing a primary amino group. Additionally, amino groups can be linked to the vector or compound containing amino groups can be linked to the vector. The linkage can be reversible or irreversible, covalent or non-covalent. Other charged groups for conferring a charge to a compound are known in the art.

In accordance with another embodiment of the disclosure a modified or mutant lambda bacteriophage is used as a recombinant viral vector. Lambda phage possesses several capsid (head) proteins. gpD (gene product D) (SEQ. ID NO. 5) is a lambda phage head protein that when in its native conformation possesses free amino and carboxy termini. As such, it is common practice to express cDNA libraries on the lambda head to investigate protein interactions, wherein the expressed cDNAs are tethered to gpD at the amino or carboxy terminus and appear on the viral head surface. In order to utilize lambda phage as an organelle delivery vehicle, the free termini of gpD are modified to contain organelle targeting signals operably linked to a protein transduction domain. Accordingly, in one embodiment a lambda bacteriophage vector is selected as a delivery vehicle wherein lambda bacteriophage specifically expresses an organelle targeting signal and protein transduction domain (PTD) present on the viral capsid. The targeting signal can be a signal sequence that specifically interacts with an organelle. The PTD can also be a signal sequence that enables the viral vector to cross cellular membranes. The PTD can be positioned in the fusion protein such that after entry into the organelle, the PTD domain is cleaved from the surface of the vector causing the vector to remain trapped in the organelle. Alternatively, cleavage sites can be engineered into the expressed polypeptide so that a desired region of the polypeptide can be cleaved within the cell, for example cleavage of the PTD once the vector is localized within the organelle. The organelle targeting signal can also be cleaved from the surface of the vector. In a preferred embodiment, the PTD sequence is followed by the organelle targeting sequence. The signal sequence can be all or part of a protein, lipid, sugar group such as a carbohydrate or a combination thereof. In this embodiment the lambda vector is introduced into the extracellular space and contacts a cell, the vector transduces across the cellular membrane and binds to its target organelle via the expressed signal sequences, and the polynucleotide present in the vector is introduced into the organelle. It will be appreciated by those skilled in the art that the target organelle can be transfected extracellularlly or intracellularly. If the target organelle is transfected extracellularly, the transfected organelle can then be introduced to a cell using techniques known in the art such as fusion, electroporation, microinjection, ballistic bombardment, or liposomes.

Another embodiment provides a method for transfecting cellular organelles, for example eukaryotic organelles, by providing a virus having a targeting signal. The targeting signal can be a polypeptide, modified or unmodified, displayed on the surface of the virus which enables the virus to specifically associate with the target organelle. Exemplary targeting signals include mitochondrial targeting signals including the targeting signals listed in TABLE 1 and other signals having a net positive charge. Contacting a cell with the recombinant vector, for example a viral vector, in a manner that introduces the vector into the cytosol of said cell as an intact functioning vector. The vector then associates with its specific target organelle and the recombinant DNA is introduced into the organelle. Introduction of the recombinant DNA into the organelle can be accomplished by transducing the vector across organelle membranes via a protein transduction domain expressed on a surface of the vector.

Introduction of a vector into the cytosol of a eukaryotic cell, in an intact functional form, can be accomplished using standard techniques known to those skilled in the art or through modification of the recombinant vector with Protein Transduction Domains. Such transfection procedures include but are not limited to microinjection, electroporation, calcium chloride premeablization, polyethylene glycol permeabilization, protoplast fusion or cationic lipid premeablization. In one embodiment a viral vector is modified to include a Protein Transduction Domain that enables the entire vector to by transduced across a lipid bilayer including a cellular membrane, organelle membrane, or plasma membrane. Suitable PTDs include but are not limited to an 11 Arginine PTD or Tat-PTD (SEQ. ID NOs. 3 or 4).

In accordance with one embodiment a method is provided for introducing exogenous nucleic acid sequences into a mitochondrion of a mammalian cell. Any mitochondrial transfection technique should ensure that a nucleic acid crosses three membranes (the plasma membrane and the outer and inner mitochondrial membranes), addresses the high copy of mtDNA molecules, and utilizes a minimal, circular mitochondrial replicon. In one embodiment of the present disclosure a recombinant bacteriophage is used as a delivery vehicle for introducing nucleic acid sequences into an organelle, for example the mitochondrion, wherein the vector does not bind to a lambda phage receptor expressed on the surface of the mitochondrion. Rather, the lambda phage vector associates with the mitochondrion via a mitochondrial targeting sequence, for example a targeting sequence of Table 1.

3.1 Transfection of Plants

Techniques for plant transfection are known in the art. For example, Agrobacterium tumefaciens and Agrobacterium rhizogenes both have the ability to transfer portions of their DNA into the genomes of plants and can be used to transfect plant cells. The mechanism by which they transfer DNA is the same, however the differences in the resulting phenotypes are attributed to the presence of a Ti plasmid in Agrobacterium tumefaciens and the Ri plasmid in Agrobacterium rhizogenes. The Ti plasmid DNA induces host plants to grow tumourous masses whereas the Ri plasmid DNA leads to the abundant proliferation of roots. Agrobacterium tumefacies is capable of infecting almost any plant tissue whereas Agrobacterium rhizogenes can only infect roots.

The Ti plasmid of Agrobacterium is a large, circular double stranded DNA molecule (T-DNA) of approximately 200 kb, which exist as an autonomous replicating unit. The plasmids are maintained within the bacteria and only a specific region (T-region) approximately 20 kb can be transferred from the bacteria to the host. To accomplish this transfer the Ti plasmid contains a series of genes that code for its own replication, excision from the plasmid, transfer to the host cell, incorporation into the host genome and the induction of tumor formation

Agrobacterium can detect and migrate towards injured plant cells through the detection of chemical signals leaking from the wounded plant. This detection process is referred to as chemotaxis. Agrobacterium can recognize plant compounds such as acetosyrinogone, sinapinic acid, coniferyl alcohol, caffeic acid and methylsyringic acid which induce the bacteria's virulence. To begin the infection process, Agrobacterium must bind itself to the host cell. This binding is achieved by a group of genes located within the bacterial chromosome. The bacteria can anchor at the site of injury, by the production of cellulose fibrils. The fibrils attach to the cell surface of the plant host and facilitate the clustering of other bacteria on the cell surface. It is believed that this clustering many help the successful transfer of T-DNA. Once bound to the host, the bacterium is free to begin the processing and transfer of the T-region. One embodiment of the present disclosure discloses transfecting a plant cell with Agrobacterium wherein the Agrobacterium has been modified to bind to a plant organgelle, for example a chloroplast. Agrobacterium can be futher modified to encode a nucleic acid of interest for expression in the organelle. Upon binding to the organelle, the Agrobacterium can deliver the target nucleic acid into the chloroplast.

To transfer the T-region of the Ti plasmid to the host cell organelle, the T-region must be processed such that it is excised from the plasmid and directed to the organelle. The T-region is excised from the Ti plasmid and directed into the host cell or organelle. Once properly packaged, the T-complex transfer is mediated by several proteins and is thought to be similar to bacterial conjugation. Once inside the plant cell or organelle, the T-complex is taken through the membrane.

4. Research Tools

In one embodiment, the present disclosure is used as a tool to investigate cellular consequences of mtDNA expression, the mechanisms of heteroplasmy, mtDNA replication and inheritance, as well as threshold effects. Mitochondrial mutant mice can be generated using this approach, allowing investigators to study mutations in mtDNA not found in nature. More particularly, the technology can be used to generate cells that contain mitochondria that have identical genotypes or varying degrees of heteroplasmy. To prepare homoplastic cells, Rho⁰ cells (devoid of mtDNA) are first prepared using RNA interference (RNAi). For example Rho⁰ cells can be generated using RNAi to the human mitochondrial DNA polymerase. Exemplary Rho⁰ cell lines are generated with RNAi to mitochondrial proteins involved in mtDNA maintenance. These Rho⁰ cells are maintained and propagated on pyruvate containing supportive media and then transfected with a functional mitochondria genome. After metabolic selection, by removing pyruvate from supportive media, only those cells that contain successfully transfected mitochondria will survive, thus generating a population of cells that all have identical mitochondria genomes.

Cell lines having varying degrees of heteroplasmy can then be generated in a controlled manner by fusing two or more homoplasmy cell lines to generate cybrids. Cybrids can be generated using any of the known technique for introducing organelles into a recipient cell, including but not limited to polyethylene glycol (PEG) mediated cell membrane fusion, cell membrane permeabilization, cell-cytoplast fusion, virus mediated membrane fusion, liposome mediated fusion, microinjection or other methods known in the art.

5. Transgenic Non-Human Animals

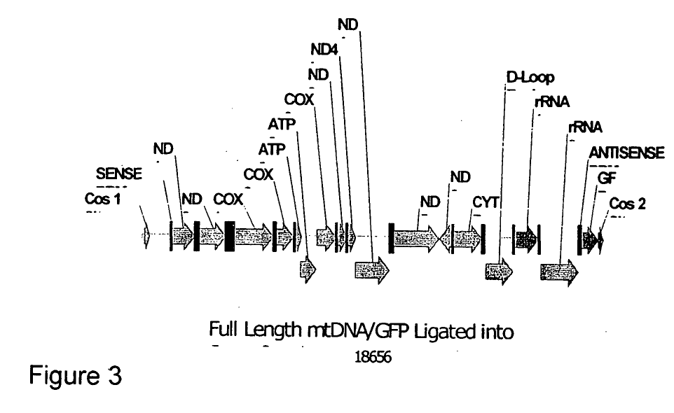

The techniques described in the present disclosure can also be used to generated transgenic non-human animals. In particular, zygote microinjection, nuclear transfers, blastomere electrofusion and blastocyst injection of embryonic stem (ES) cell cybrids have each provided feasible strategies for creating hetero- and homoplasmic mice containing mtDNA from mitofected cell lines (i.e. cells that containing transfected mitochondria). In one embodiment an embryonic stem (ES) cell is mitofected and injected into the blastocyst of a mammalian embryo as a means of generating chimeric mice. In another embodiment, embryonic stem (ES) cell cybrids (from mitofected cells and ES cell rhos, or from two separately mitofected cells) are first prepared, followed by blastocyst injection into embryos as shown in FIG. 3. The use of cells carrying specific mitofected mtDNA of interest allows the creation of transmitochondrial mice that are heteroplasmic or even homoplasmic for the mitofected DNA. In theory, this technique offers the prospect of transferring any mutant mtDNA that can be obtained from cultured mitofected cells into a whole organism model.

Using lambda for mtDNA transfection will allow investigations into questions such as the effect of varying proportions of the 5000 bp “common deletion”, which accumulates with aging, polymorphisms found in diabetes and neurodegenerative diseases, and dynamics of mtDNA complementation. There are also potential therapeutic uses of this approach. Targeted introduction of the normal mitochondrial genome offers treatment for both classic mtDNA-based diseases and diseases of aging such as neurodegenerative brain conditions and adult-onset diabetes, which have been associated with mtDNA-based mitochondrial dysfunction.

6. Kits

The present disclosure is also directed to a kit or pack that supplies the elements necessary to conduct transfection of eukaryotic organelles. In accordance with one embodiment a kit is provided comprising a protein construct, that encodes an organelle localizing signal and protein transduction domain operably linked to a lambda surface protein, and lambda packaging components for preparing a recombinant lambda vector. The kit may also include the lambda DNA sequences (the vector “arms”) for inserting a DNA sequence of interest and subsequent use in generating a recombinant lambda phage vector. In one embodiment the protein construct provided with the kit comprises a mitochondrial or chloroplast localization signal selected from those known to target to the organelle, partially listed in Tables I and II, and more particularly in one embodiment the protein construct comprises a sequence encoding a 11 Arginine stretch followed by the mitochondrial localization signal of subunit VIII of human cytochrome oxidase operably linked to the bacteriophage lambda gpD head protein.

In accordance with one embodiment a kit is provided comprising cells that contain either a mitochondria or chloroplast organelle that expresses an exogenous nucleic acid. In a further embodiment a kit is provided that comprises packaging components for a viral vector including the recombinant PTD-organelle targeting signal surface protein and viral DNA for preparing recombinant constructs. In one embodiment the kit is provided with recombinant PTD-organelle targeting signal lambda surface protein, lambda bacteriophage packaging extract, and lambda DNA. The individual components of the kits can be packaged in a variety of containers, e.g., vials, tubes, microtiter well plates, bottles, and the like. Other reagents can be included in separate containers and provided with the kit; e.g., positive control samples, negative control samples, buffers, cell culture media, etc. Preferably, the kits will also include instructions for use.

7. Methods of Treatment

Organelle dysfunction can cause disease in a host, for example a human host or a plant host. In particular, problems with mitochondria or chloroplasts can result in disease. Mitochondrial diseases result from failures of the mitochondria, specialized compartments present in every cell of the body except red blood cells. Cell injury and even cell death are result from mitochondrial failure. If this process is repeated throughout the body, whole systems begin to fail, and the life of the person in whom this is happening is severely compromised. The disease can be in children, for example individuals less that 18 years of age, typically less than 12 years of age, or adults, for example individuals 18 years of age or more. Thus, embodiments of the present disclosure are directed to treating a host diagnosed with an organelle related disease, in particular a mitochondrial disease, by introducing a vector into the host cell wherein the vector specifically binds to the organelle and wherein the vector comprises a nucleic acid encoding mitochondrial protein or peptide. The present disclosure encompasses manipulating, augmenting or replacing portions of the mammalian cell mitochondrial genome to treat diseases caused by mitochondrial genetic defects or abnormalities.

Exemplary mitochondrial diseases include but are not limited to: Alpers Disease; Barth syndrome; ã-oxidation defects; carnitine-acyl-carnitine deficiency; carnitine deficiency; co-enzyme Q10 deficiency; Complex I deficiency; Complex II deficiency; Complex III deficiency; Complex IV deficiency; Complex V deficiency; cytochrome c oxidase (COX) deficiency; Chronic Progressive External Ophthalmoplegia Syndrome (CPEO); CPT I Deficiency; CPT II deficiency; Glutaric Aciduria Type II; lactic acidosis; Long-Chain Acyl-CoA Dehydrongenase Deficiency (LCAD); LCHAD; mitochondrial cytopathy; mitochondrial DNA depletion; mitochondrial encephalopathy; mitochondrial myopathy; Mitochondrial Encephalomyopathy with Lactic Acidosis and Strokelike episodes (MELAS); Myoclonus Epilepsy with Ragged Red Fibers (MERRF); Maternally Inherited Leigh's Syndrome (MILS); Myogastrointestinal encephalomyopathy (MNGIE); Neuropathy, ataxia and retinitis pigmentosa (NARP); Leber's Hereditary Optic Neuropathy (LHON); Progressive external ophthalmoplegia (PEO); Pearson syndrome; Keams-Sayre syndrome (KSS); Leigh's syndrome; intermittent dysautonomia; pyruvate carboxylase deficiency; pyruvate dehydrogenase deficiency; respiratory chain mutations and deletions; Short-Chain Acyl-CoA Dehydrogenase Deficiency (SCAD); SCHAD; and Very Long-Chain Acyl-CoA Dehydrongenase Deficiency (VLCAD).

Some mitochondrial diseases are a result of problems in the respiratory chain in the mitochondira. The respiratory chain consists of four large protein complexes: I, II, III and IV (cytochrome c oxidase, or COX), ATP synthase, and two small molecules that ferry around electrons, coenzyme Q10 and cytochrome c. The respiratory chain is the final step in the energy-making process in the mitochondrion where most of the ATP is generated. Mitochondrial encephalomyopathies that can be caused by deficiencies in one or more of the specific respiratory chain complexes include MELAS, MERFF, Leigh's syndrome, KSS, Pearson, PEO, NARP, MILS and MNGIE.

The mitochondrial respiratory chain is made up of proteins that come from both nuclear and mtDNA. Although only 13 of roughly 100 respiratory chain proteins come from the mtDNA, these 13 proteins contribute to every part of the respiratory chain except complex II, and 24 other mitochondrial genes are required just to manufacture those 13 proteins. Thus, a defect in either a nuclear gene or one of the 37 mitochondrial genes can cause the respiratory chain to break down. It will be appreciated that the scope of the present disclosure includes transfecting mitochondria with at least one or part of one gene involved in mitochondrial function, in particular at least one or part of the 37 mitochondrial genes to restore or increase the function of the respiratory chain. Any or part of a mitochondrial genome, for example human mitochondrial genome SEQ ID NO: 6, may be introduced into a host mitochondrion using the methods described herein.

Diseases of the mitochondria appear to cause the most damage to cells of the brain, heart, liver, skeletal muscles, kidney and the endocrine and respiratory systems. Thus, transfection of mitochondria in these cells and tissues with specific nucleic acids is within the scope of the present disclosure, in particular transfection of mitochondria with nucleic acids encoding mitochondrial-encoded proteins rather than nuclear-encoded proteins. It will be appreciated that the mitochondria can be transfected to express any protein whether naturally present in the mitochondrion or not or naturally encoded by mtDNA or nuclear DNA. Depending on which cells are affected, symptoms may include loss of motor control, muscle weakness and pain, gastro-intestinal disorders and swallowing difficulties, poor growth, cardiac disease, liver disease, diabetes, respiratory complications, seizures, visual/hearing problems, lactic acidosis, developmental delays and susceptibility to infection.

Exmplary mtDNA mutations that can be addressed by the present disclosure include but are not limited to: tRNA^{leu}-A3243G, A3251G, A3303G, T3250C T3271C and T3394C; tRNA^{Lys}-A8344G, G11778A, G8363A, T8356C; ND1-G3460A; ND4-A10750G, G14459A; ND6-T14484A; 12S rRNA-A1555G; MTTS2-C12258A; ATPase 6-T8993G, T8993C; tRNA^{Ser}(UCN)-T7511C; 11778 and 14484, LHON mutations as well as mutations or deletions in ND2, ND3, ND5, cytochrome b, cytochrome oxidase I-III, and ATPase 8.

One embodiment of the present disclosure provides a method for restoring or increasing respiratory chain function in a host cell including introducing a vector into the host cell, wherein the vector specifically binds to the mitochondrion and comprises a nucleic acid that encodes a respiratory chain protein or peptide. The nucleic acid of the vector can be injected or otherwise delivered into the interior of the mitochondria when the vector targets the mitochondria, for example when the vector is bacteriophage lambda and the viral surface protein is gpD operably linked to a protein transduction domain and mitochondrial localization signal.

Another embodiment of the present disclosure provides a method for restoring or increasing cytochrome oxidase activity in a host including transfecting mitochondria in a cell, for example a skeletal muscle cell, wherein the vector comprises a nucleic acid that encodes cytochrome oxidase or a functional component thereof. A functional component means a part or fragment of the protein or protein complex or subunit that performs a biological function independently or in combination with another protein, fragment, or subunit.

Still another embodiment of the present disclosure provides a method of increasing or restoring β-oxidation in a host including obtaining cells from the host, transfecting an organelle in the cells from the host, introducing a vector comprising a nucleic acid encoding proteins involved in β-oxidation spiral and carnitine transport, wherein the vector specifically binds to the organelle; and introducing the transfected cells of the host back into the host. Other embodiments of the disclosure are directed to methods of restoring mitochondrial function lost or decreased as a result of point mutations or deletions. For example, KSS, PEO and Pearson, are three diseases that result from a type of mtDNA mutation called a deletion (specific portions of the DNA are missing) or mtDNA depletion (a general shortage of mtDNA). Thus, cells from hosts diagnosed with KSS, PEO, Pearson or similar disease can have their mitochondria transfected with the recombinant viral vector. A vector comprising a nucleic acid that corresponds to the deletion in the mtDNA causing the diseased state can be introduced into the cells. The vector will bind the organelle and deliver the nucleic acid into the interior of the mitochondria where the nucleic acid is expressed. The expression product can then incorporate into the mitochondria and increase or restore mitochondrial function. The transfected cells can be reintroduced in the host. It will be appreciated that the host's cells or other cells can be transfected as described herein and introduced into a host having a dysfunctional organelles, in particular mitochondria.

It will be appreciated by those skilled in the art that the present disclosure encompasses delivering either separately or in combination nucleic acids to the mitochondria that are naturally encoded by mtDNA or nuclear DNA.

The present disclosure also contemplates alleviating the symptoms of mitochondrial diseases by creating cells having tranfected and non-transfected mitochondria. Alternatively, all of the mitochondria in a cell can be transfected or replaced.

One embodiment provides a method for compensating for a mtDNA mutation in a host, the method including identifying a host having a mtDNA mutation, obtaining a cell comprising said mtDNA mutation from said host, transfecting a mitochondrion of the host cell, introducing a vector that specifically binds to the organelle into the host cell, wherein the vector comprises a nucleic acid that encodes a functional product corresponding to the mtDNA mutation, introducing said transfected cell into the host. A nucleic acid that encodes a functional product corresponding to the mtDNA mutation means a sequence that produces a protein without the corresponding mutation. For example, if a host cell has an ND4-A10750G mutation, the transfected nucleic acid would encode a wildtype product for the ND4 gene. The viral vector can be introduced into the host, for example, intravenously.

8. Depletion of Oganelle-Specific Polynucleotides

Another embodiment provides a method for depleting organelle polynucleotides, for example mitochondrial or chlorpolast polynucleotides. The method includes contacting a cell with at least one inhibitory nucleic acid, for example siRNA, specific for a polymerase, for example an organelle specific polymerase. One exemplary polymerase is POLγ. The inhbitory nucleic acid can be selected from sequences listed in TABLE 3 or a sequence having 80-100% homology to the sequences listed in TABLE 3. The inhibitor nucleic acid can be delivered to the organelle of interest using the compositions disclosed herein or using conventional transfection techniques.

Still another embodiment provides a method of depleting mtDNA in a cell including contacting the cell with an inhibitor of mtDNA replication or transcription. The inhibitor of mtDNA replication can be an antisense polynucleotide or a small inhibitory RNA, or a combination thereof. It will be appreciated that the inhibitor can be DNA, RNA or a combination thereof. Exemplary inhibitors are selected from Table 3.

In some embodiments, the inhibitor is specific for a gene involved in mitochondral DNA transcription or replication. Exemplary genes include, but are not limited to, Polγ, TFAM A and B, and mtSSB. Generally, genes that encode a mitochondrial or chloroplast polymerase can be used.

Another embodiment provides a cell having an inhibitory polynucleotide that specifically binds to a polynucleotide encoding a mitochondrial polymerase. The cell can contain an siRNA specific for a mitochondrial polymerase, for example Polγ or a vector that expresses the inhibitory polynucleotide.

9. Administration

The compositions provided herein may be administered in a physiologically acceptable carrier to a host. Preferred methods of administration include systemic or direct administration to a cell. The compositions can be administered to a cell or patient, as is generally known in the art for gene therapy applications. In gene therapy applications, the compositions are introduced into cells in order to transfect an organelle. “Gene therapy” includes both conventional gene therapy where a lasting effect is achieved by a single treatment, and the administration of gene therapeutic agents, which involves the one time or repeated administration of a therapeutically effective DNA or RNA.

The modified vector compositions can be combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as Tween, Pluronics or PEG.

The compositions of the present disclosure can be administered parenterally. As used herein, “parenteral administration” is characterized by administering a pharmaceutical composition through a physical breach of a subject's tissue. Parenteral administration includes administering by injection, through a surgical incision, or through a tissue-penetrating non-surgical wound, and the like. In particular, parenteral administration includes subcutaneous, intraperitoneal, intravenous, intraarterial, intramuscular, intrasternal injection, and kidney dialytic infusion techniques.

Parenteral formulations can include the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Parenteral administration formulations include suspensions, solutions, emulsions in oily or aqueous vehicles, pastes, reconsitutable dry (i.e. powder or granular) formulations, and implantable sustained-release or biodegradable formulations. Such formulations may also include one or more additional ingredients including suspending, stabilizing, or dispersing agents. Parenteral formulations may be prepared, packaged, or sold in the form of a sterile injectable aqueous or oily suspension or solution. Parenteral formulations may also include dispersing agents, wetting agents, or suspending agents described herein. Methods for preparing these types of formulations are known. Sterile injectable formulations may be prepared using non-toxic parenterally-acceptable diluents or solvents, such as water, 1,3-butane diol, Ringer's solution, isotonic sodium chloride solution, and fixed oils such as synthetic monoglycerides or diglycerides. Other parentally-administrable formulations include microcrystalline forms, liposomal preparations, and biodegradable polymer systems. Compositions for sustained release or implantation may include pharmaceutically acceptable polymeric or hydrophobic materials such as emulsions, ion exchange resins, sparingly soluble polymers, and sparingly soluble salts.

Pharmaceutical compositions may be prepared, packaged, or sold in a buccal formulation. Such formulations may be in the form of tablets, powders, aerosols, atomized solutions, suspensions, or lozenges made using known methods, and may contain from about 0.1% to about 20% (w/w) active ingredient with the balance of the formulation containing an orally dissolvable or degradable composition and/or one or more additional ingredients as described herein. Preferably, powdered or aerosolized formulations have an average particle or droplet size ranging from about 0.1 nanometers to about 200 nanometers when dispersed.

As used herein, “additional ingredients” include one or more of the following: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other “additional ingredients” which may be included in the pharmaceutical compositions are known. Suitable additional ingredients are described in Remington's Pharmaceutical Sciences, Mack Publishing Co., Genaro, ed., Easton, Pa. (1985).

Dosages and desired concentrations of modified vectors disclosed herein in pharmaceutical compositions of the present disclosure may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

### EXAMPLES

### Example 1

Modification of the Bacteriophage Lambda Head Protein D

gpD (gene product D) was PCR amplified using the Stratagene Hotstart Herculase PCR polymerase from Lambda genome (NEB) with the following forward and reverse primers designed to contain BgIII and NotI ends, respectively. Of note, the stop codon in the reverse oligonucleotide was removed. The cDNA was digested with BgIII and NotI (NEB) and cloned into the BgIII and NotI sites of pThioHisA (Stratagene). The resultant vector was named pThio-gpD. A start Methionine and subsequent 11 Arginine containing forward oligonucleotide was designed for PCR amplification of the mitochondrial localized Discosoma Red Fluorescent Protein (RFP) from pDsRed2-Mito (Clontech). The forward oligonucleotide also carried a 5′ KpnI site. The reverse oligonucleotide was designed to remove the stop codon from the mitochondrially localized RFP and a 5′ BgIII site was introduced. PCR amplification using Hotstart Herculase

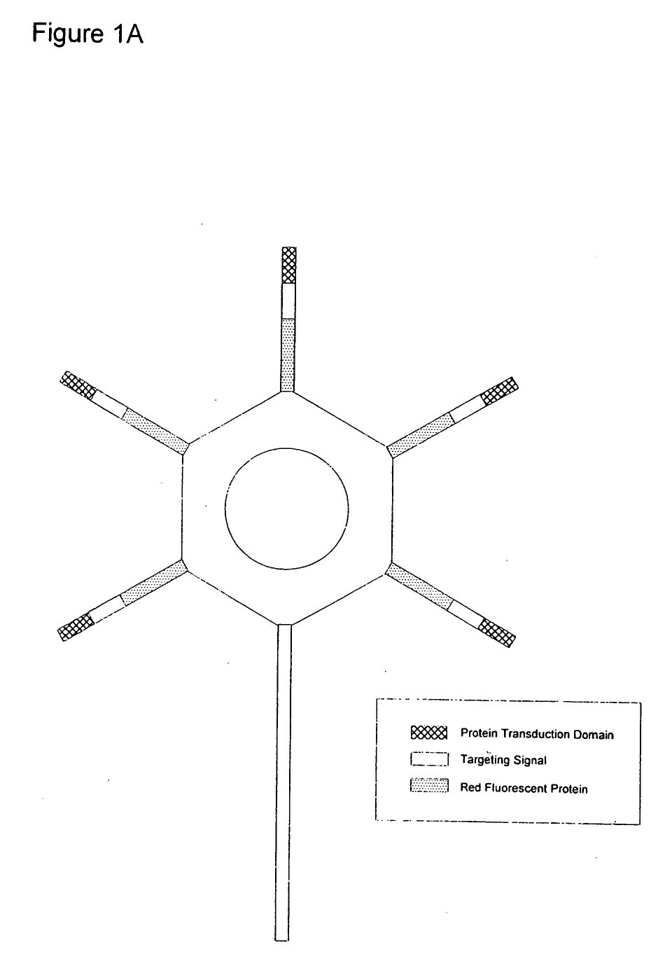

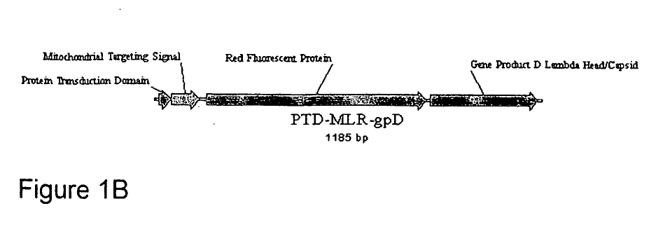

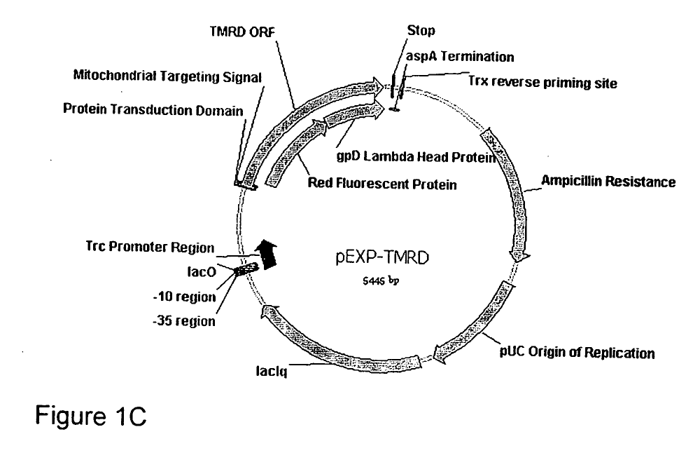

PCR polymerase was carried out on pDsRed2-Mito using the forward and reverse primers mentioned to generate a cDNA containing a 5′ KpnI site followed by a start Methionine codon, 11 Arginine codons, the cDNA for the mitochondrially localized RFP ending in a 3′ BgIII site. The cDNA construct was digested with KpnI and BgIII and cloned into pThio-gpD digested with KpnI and BgIII. The resultant plasmid was named pEXP-TMRD (Expression plasmid for Transducing Mitochondrial Red fluorescent protein gpD) (FIG. 1C).

pEXP-TMRD was used to transform DH5Ã cells. Colonies were isolated and sequenced for the proper plasmid. The colony containing the correct construct was grown in 10 milliliter culture to an optical density value of 0.5. IPTG was added to 1 mM to induce expression of the TMRD protein. The culture was incubated for 4 hours in a 37° C. shaking incubator. The bacterial culture was centrifuged and the resulting pellet frozen overnight at −80 C. The pellet was resuspended in 5 ml BPER lysis reagent (Pierce). Lysozyme, 100 ul of 10 mg/ml stock solution (Pierce), was added to the suspension to a final concentration of 200 ug/ml. The resultant mixture was incubated at room temperature for 5 minutes. 15 ml of 1:10 diluted BPER reagent were added to the suspension, and mixed by vortexing. In order to collect the inclusion bodies containing the TMRD protein, centrifugation at 27,000 g was done for 15 minutes. The pellet was resuspended in 20 ml of 1:10 diluted BPER Reagent. The centrifugation and pellet resuspension steps were repeated two more times. The inclusion bodies were solubilized with 7 M urea, 1 mM DTT, 50 mM Tris.HCl (pH 8.0), and 150 mM NaCl. The solubilized inclusion bodies were dialyzed against saline containing 33% PBS overnight. Further purification was carried out using the HisPatch Thiofusion S.N.A.P. purification columns (Invitrogen) following manufacture's directions. The resulting protein concentrate was incubated with enterokinase at 1 mg/ml overnight at room temperature. Protein concentration was quantified with the Lowry assay (Biorad). Purified TMRD was introduced into the GigaPack Gold packaging extract (Stratagene) to a final concentration of 0.5 mg/ml. The final lambda packaging extract containing TMRD in excess was used immediately.

### Example 2

Transfection of Mitochondria in Living Cells and Expression of a Recombinant Gene Construct

Through PCR, a full-length mitochondrial genome using established primers to amplify the entire mitochondrial genome sequence of Sy5y cells was generated. The full-length mtDNA was digested with BgIII and NotI. Once digested, a NotI, BgIII digested GFP was ligated to mtDNA amplicon. To this larger molecule, the Cos sites were ligated to generate a packageable construct. The cloning strategy is outlined below.

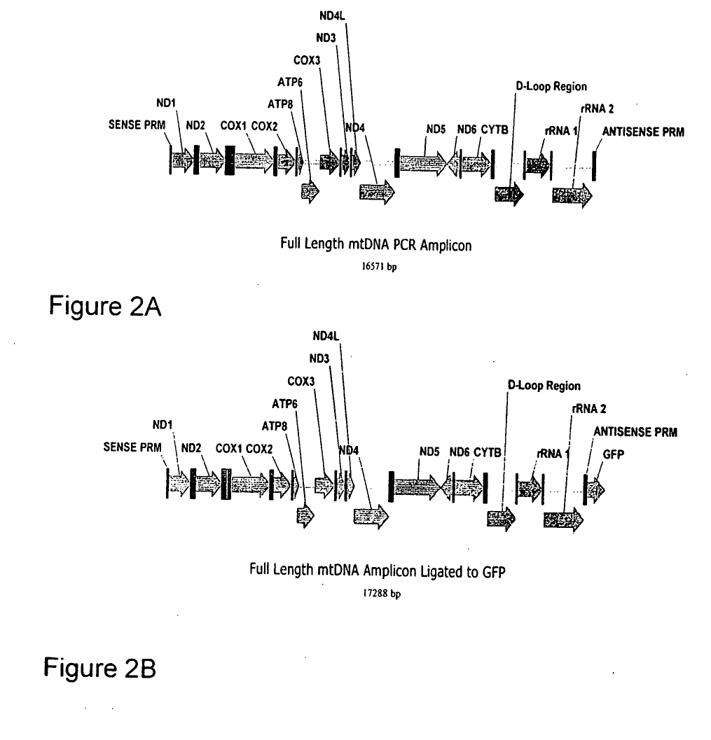

A full length mtDNA PCR amplicon is generated with sense and anti-sense primers containing internal BgIII and NotI sites, respectively, and digested with BgIII and NotI (FIG. 2A). A GFP DNA will be excised from its vector, pEGFP-1, using BgIII and NotI sites. This GFP construct will be ligated to the mtDNA amplicon generated by PCR and digested. The resulting molecule may contain both 5′ and 3′ sense GFPs (FIG. 2B).

A GFP that has been mutated to be expressed only in mitochondria was used. Briefly, the 60th codon of the GFP mRNA is a UGG—it is read as a tryptophan by both the nuclear and mitochondrial translation apparatus. By mutating this codon to a UGA generates a stop if translated by the nucleus, but is translated as a tryptophan in the mitochondria. This strategy takes advantage of the difference between mitochondrial and nuclear codons producing a curtailed non-fluorescing protein if translated in the ER otherwise producing a full length GFP in the mitochondria. This GFP construct was ligated to the full length mtDNA amplicon (FIG. 3).

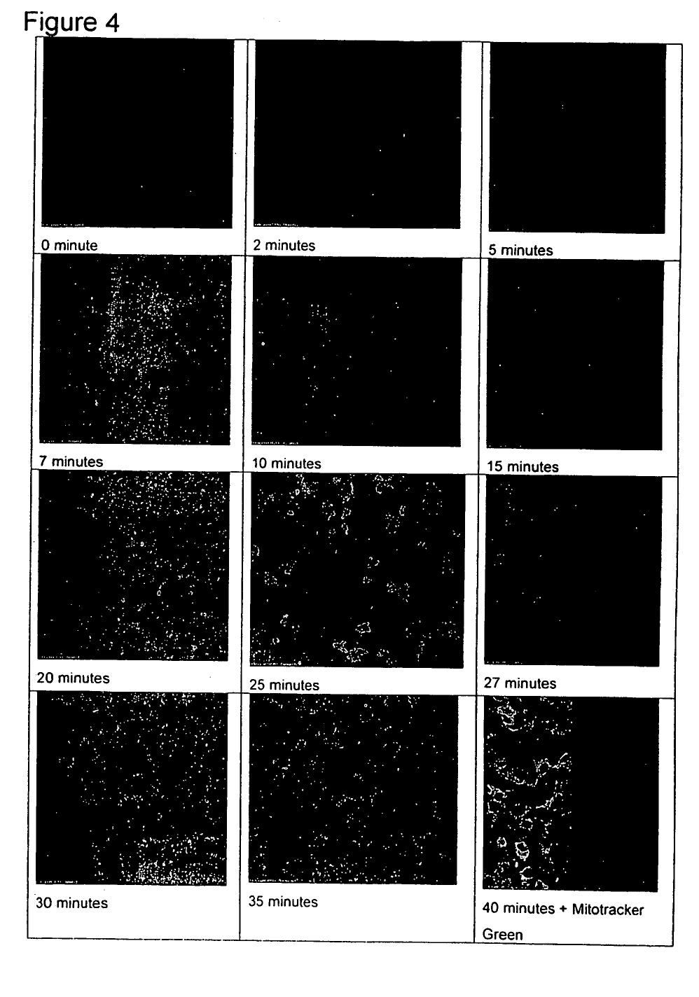

Once the construct illustrated in FIG. 3 was packaged with packaging extract including the recombinant PTD-organelle targeting signal capsid protein, the active phage particles were introduced into the extracellular media of cells devoid of mtDNA generated with RNAi. Since the recombinant capsid protein contains a reporter gene, RFP, confocal microscopy was used to follow and verify the location of the recombinant viral vector (FIG. 4). The 40-minute micrograph includes mitochondria specific dye, Mitotracker Green (Molecular Probes) to verify mitochondrial localization.

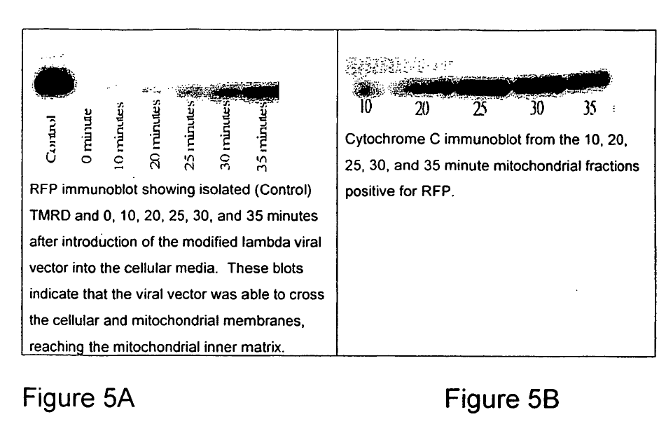

Mitochondrial targeting was further verified with western blotting of mitochondrial fractions generated at specific time points using antibodies to RFP (Clontech) and Cytochrome C (Pharmingen) (FIG. 5). A mitochondrial fraction was prepared from Sy5y cells in culture at several time points after introducing the modified bacteriophage lambda viral vector and immunoblotted with an anti-RFP antibody (Clontech). The same mitochondrial fractions that contained an anti-RFP signal were probed with an anti-body for cytochrome C, an endogenous mitochondrial matrix protein. RFP immunoblot showing isolated (Control) TMRD and 0, 10, 20, 25, 30, and 35 minutes after introduction of the modified lambda viral vector into the cellular media. These blots indicate that the viral vector was able to cross the cellular and mitochondrial membranes, reaching the mitochondrial inner matrix. Cytochrome C immunoblot from the 10, 20, 25, 30, and 35 minute mitochondrial fractions positive for RFP.

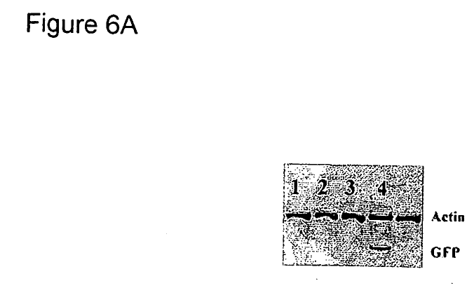

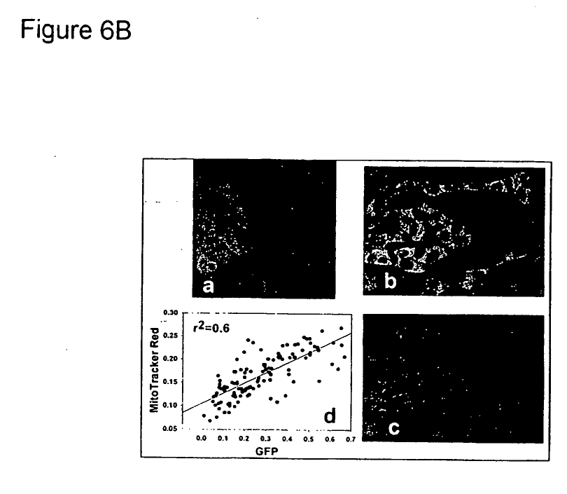

FIG. 6A shows the successful introduction of lambdaphage targeted to mitochondria. FIG. 6A shows GFP message in mitochondria (demonstrated with RT-PCR). This approach yields very efficient GFP expression in mitochondria, shown by the correlation graph in FIG. 6d. Thus successfully demonstrated the ability of the technology to reintroduce intact mitochondrial genomes into rho⁰ SY5Y cells. FIG. 6B shows (a) appearance of ρ0 cells 24 hours after transfection with RFP recombinant phage; (b) appearance of ρ0 cells transfected with SuperCos-1/mtDNA/GFP recombinant phage construct under green fluoresence after 24 hours; (c) companion images of MitoTracker Red staining (c) to reveal location of mitochondria. Panel (d) is a scatterplot comparing fluorescence intensities among MitoTracker Red mitochondrial clusters and GFP reporter gene expression.

### Example 3

siRNA Knockdown of PolG

RNA interference (RNAi) is based on an evolutionarily conserved mechanism to prevent replication and expression of exogenous double-stranded RNA. While initially described in plants, RNAi has now been described in eukaryotic, vertebrate, and mammalian cells. The basic mechanism depends on the generation of small RNA duplexes of 21-23 nucleotides in length and with 2-3 nucleotide overhangs at each end. In cells infected with exogenous RNA viruses, these small interfering RNA's (siRNA's) are produced by the action of a RNAase complex known as “dicer”. The siRNA's thus generated (or supplied as exogenous duplexes or generated internally from vectors) then form an RNA—induced silencing complex (RISC), which utilizes the anti-sense RNA strand to hybridize to the specific mRNA gene product. RISC then can create a new RNA duplex that dicer or perhaps RISC itself can degrade.

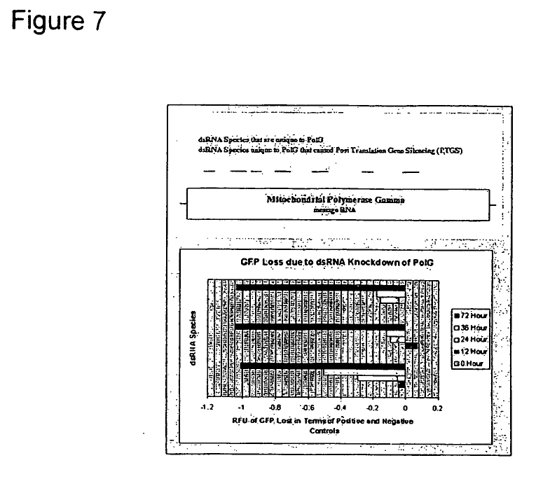

RNA silencing of mitochondrial polymerase POIG occurs within 3 days. Rho⁰ cell lines were created by chronic incubation with the mutagen ethidium bromide to provide a slow mutagenesis and depletion of mtDNA. To develop more useful cell lines, a molecular RNA interference approach to silence the POIG gene was utilized and described here for the first time. The results of this experiment are shown in FIG. 7. RNA silencing of the POLG gene. (top) Locations of unique sequences in POL-γ mRNA chosen for RNA duplex construction. Three different duplexes were synthesized and tested. (bottom) complete loss of GFP fluorescence over 72 hrs after lipofection of RNA duplexes, indirectly indicating silencing of POLG.

The PolG gene (SEQ ID NO.: 195) was examined and several apparently unique sequences were defined to create appropriate siRNA duplexes and exemplary target sequences and siRNA sequences are provided in Table 3. Representative siRNAs were introduced by lipophilic amine into cells transfected with the recombinant lambda vector. Because mtDNA synthesis and transcription are so closely linked, the loss of GFP signal was followed as a marker for loss of PolG activity (FIG. 7).

TABLE 3
PoIG siRNA Sequences

| | |
|---|---|
| Target sequence 1: AAGGTGGCCGGCGCCACCGTC | (SEQ ID NO. 196) |
| Position in gene sequence: 22 | |
| GC content: 76.2% | |
| Sense strand siRNA: GGUGGCCGGCGCCACCGUCtt | (SEQ ID NO. 197) |
| Antisense strand siRNA: GACGGUGGCGCCGGCCACCtt | (SEQ ID NO. 198) |
| Target sequence 2: AACAGCAGCCTCAGCAGCCGC | (SEQ ID NO. 199) |
| Position in gene sequence: 158 | |
| GC content: 66.7% | |
| Sense strand siRNA: CAGCAGCCUCAGCAGCCGCtt | (SEQ ID NO. 200) |
| Antisense strand siRNA: GCGGCUGCUGAGGCUGCUGtt | (SEQ ID NO. 201) |
| Target sequence 3: AAGTGCTATCCTCGGAGGGCG | (SEQ ID NO. 202) |
| Position in gene sequence: 179 | |
| GC content: 61.9% | |
| Sense strand siRNA: GUGCUAUCCUCGGAGGGCGtt | (SEQ ID NO. 203) |
| Antisense strand siRNA: CGCCCUCCGAGGAUAGCACtt | (SEQ ID NO. 204) |
| Target sequence 4: AACCCATTGGACATCCAGATG | (SEQ ID NO. 205) |
| Position in gene sequence: 214 | |
| GC content: 47.6% | |
| Sense strand siRNA: CCCAUUGGACAUCCAGAUGtt | (SEQ ID NO. 206) |
| Antisense strand siRNA: CAUCUGGAUGUCCAAUGGGtt | (SEQ ID NO. 207) |
| Target sequence 5: AAATCTTCGGGCAAGGAGGGG | (SEQ ID NO. 208) |
| Position in gene sequence: 257 | |
| GC content: 57.1% | |
| Sense strand siRNA: AUCUUCGGGCAAGGAGGGGtt | (SEQ ID NO. 209) |
| Antisense strand siRNA: CCCCUCCUUGCCCGAAGAUtt | (SEQ ID NO. 210) |
| Target sequence 6: AAGGAGGGGAGATGCCTGGCG | (SEQ ID NO. 211) |
| Position in gene sequence: 269 | |
| GC content: 66.7% | |
| Sense strand siRNA: GGAGGGGAGAUGCCUGGCGtt | (SEQ ID NO. 212) |
| Antisense strand siRNA: CGCCAGGCAUCUCCCCUCCtt | (SEQ ID NO. 213) |
| Target sequence 7: AAGCACGGGCTCTGGGGGCAG | (SEQ ID NO. 214) |
| Position in gene sequence: 325 | |
| GC content: 71.4% | |
| Sense strand siRNA: GCACGGGCUCUGGGGGCAGtt | (SEQ ID NO. 215) |
| Antisense strand siRNA: CUGCCCCCAGAGCCCGUGCtt | (SEQ ID NO. 216) |
| Target sequence 8: AACCTGGACCAGCACTTCCGC | (SEQ ID NO. 217) |
| Position in gene sequence: 400 | |
| GC content: 61.9% | |
| Sense strand siRNA: CCUGGACCAGCACUUCGGCtt | (SEQ ID NO. 218) |
| Antisense strand siRNA: GCGGAAGUGCUGGUCCAGGtt | (SEQ ID NO. 219) |
| Target sequence 9: AAGCAGAGCCTGCCCTACCTG | (SEQ ID NO. 220) |
| Position in gene sequence: 433 | |
| GC content: 61.9% | |
| Sense strand siRNA: GCAGAGCCUGCCCUACCUGtt | (SEQ ID NO. 221) |
| Antisense strand siRNA: CAGGUAGGGCAGGCUCUGCtt | (SEQ ID NO. 222) |
| Target sequence 10: AACTTGCTGTTGCAGGCCCAG | (SEQ ID NO. 223) |
| Position in gene sequence: 463 | |
| GC content: 57.1% | |
| Sense strand siRNA: CUUGCUGUUGCAGGCCCAGtt | (SEQ ID NO. 224) |
| Antisense strand siRNA: CUGGGCCUGCAACAGCAAGtt | (SEQ ID NO. 225) |
| Target sequence 11: AAGCCCCCGGCTTGGGCCTGG | (SEQ ID NO. 226) |
| Position in gene sequence: 493 | |
| GC content: 76.2% | |
| Sense strand siRNA: GCCCCCGGCUUGGGCCUGGtt | (SEQ ID NO. 227) |
| Antisense strand siRNA: CCAGGCCCAAGCCGGGGGCtt | (SEQ ID NO. 228) |
| Target sequence 12: AACTTGCCCCACATTGGCGGT | (SEQ ID NO. 229) |
| Position in gene sequence: 618 | |
| GC content: 57.1% | |
| Sense strand siRNA: CUUGCCCCACAUUGGCGGUtt | (SEQ ID NO. 230) |
| Antisense strand siRNA: ACCGCCAAUGUGGGGCAAGtt | (SEQ ID NO. 231) |
| Target sequence 13: AAGAGCGTTACTCTTGGACCA | (SEQ ID NO. 232) |
| Position in gene sequence: 689 | |
| GC content: 47.6% | |
| Sense strand siRNA: GAGCGUUACUCUUGGACCAtt | (SEQ ID NO. 233) |
| Antisense strand siRNA: UGGUCCAAGAGUAACGCUCtt | (SEQ ID NO. 234) |
| Target sequence 14: AATGTTTCCTTTGACCGAGCT | (SEQ ID NO. 235) |
| Position in gene sequence: 808 | |
| GC content: 42.9% | |
| Sense strand siRNA: UGUUUCCUUUGACCGAGCUtt | (SEQ ID NO. 236) |
| Antisense strand siRNA: AGCUCGGUCAAAGGAAACAtt | (SEQ ID NO. 237) |
| Target sequence 15: AAGCAGCTTCCAGCGCAGTCT | (SEQ ID NO. 238) |
| Position in gene sequence: 912 | |
| GC content: 57.1% | |
| Sense strand siRNA: GCAGCUUCCAGCGCAGUCUtt | (SEQ ID NO. 239) |
| Antisense strand siRNA: AGACUGCGCUGGAAGCUGCtt | (SEQ ID NO. 240) |
| Target sequence 16: AAGCAGGGCAAACACAAGGTC | (SEQ ID NO. 241) |
| Position in gene sequence: 946 | |
| GC content: 52.4% | |
| Sense strand siRNA: GCAGGGCAAACACAAGGUCtt | (SEQ ID NO. 242) |
| Antisense strand siRNA: GACCUUGUGUUUGCCCUGCtt | (SEQ ID NO. 243) |
| Target sequence 17: AAACACAAGGTCCAGCCCCCC | (SEQ ID NO. 244) |
| Position in gene sequence: 955 | |
| GC content: 61.9% | |
| Sense strand siRNA: ACACAAGGUCCAGCCCCCCtt | (SEQ ID NO. 245) |
| Antisense strand siRNA: GGGGGGCUGGACCUUGUGUtt | (SEQ ID NO. 246) |
| Target sequence 18: AAGGTCCAGCCCCCCACAAAG | (SEQ ID NO. 247) |
| Position in gene sequence: 961 | |
| GC content: 61.9% | |
| Sense strand siRNA: GGUCCAGCCCCCCACAAAGtt | (SEQ ID NO. 248) |
| Antisense strand siRNA: CUUUGUGGGGGGCUGGACCtt | (SEQ ID NO. 249) |
| Target sequence 19: AAAGCAAGGCCAGAAGTCCCA | (SEQ ID NO. 250) |
| Position in gene sequence: 978 | |
| GC content: 52.4% | |
| Sense strand siRNA: AGCAAGGCCAGAAGUCCCAtt | (SEQ ID NO. 251) |
| Antisense strand siRNA: UGGGACUUCUGGCCUUGCUtt | (SEQ ID NO. 252) |
| Target sequence 20: AAGGCCAGAAGTCCCAGAGGA | (SEQ ID NO. 253) |
| Position in gene sequence: 983 | |
| GC content: 57.1% | |
| Sense strand siRNA: GGCCAGAAGUCCCAGAGGAtt | (SEQ ID NO. 254) |
| Antisense strand siRNA: UCCUCUGGGACUUCUGGCCtt | (SEQ ID NO. 255) |
| Target sequence 21: AAGTCCCAGAGGAAAGCCAGA | (SEQ ID NO. 256) |
| Position in gene sequence: 991 | |
| GC content: 52.4% | |
| Sense strand siRNA: GUCCCAGAGGAAAGCCAGAtt | (SEQ ID NO. 257) |
| Antisense strand siRNA: UCUGGCUUUCCUCUGGGACtt | (SEQ ID NO. 258) |
| Target sequence 22: AAAGCCAGAAGAGGCCCAGCG | (SEQ ID NO. 259) |
| Position in gene sequence: 1003 | |
| GC content: 61.9% | |
| Sense strand siRNA: AGCCAGAAGAGGCCCAGCGtt | (SEQ ID NO. 260) |
| Antisense strand siRNA: CGCUGGGCCUCUUCUGGCUtt | (SEQ ID NO. 261) |
| Target sequence 23: AAGAGGCCCAGCGATCTCATC | (SEQ ID NO. 262) |
| Position in gene sequence: 1011 | |
| GC content: 57.1% | |
| Sense strand siRNA: GAGGCCCAGCGAUCUCAUCtt | (SEQ ID NO. 263) |
| Antisense strand siRNA: GAUGAGAUCGCUGGGCCUCtt | (SEQ ID NO. 264) |
| Target sequence 24: AACAGTCTGGCAGAGGTGCAC | (SEQ ID NO. 265) |
| Position in gene sequence: 1060 | |
| GC content: 57.1% | |
| Sense strand siRNA: CAGUCUGGCAGAGGUGCACtt | (SEQ ID NO. 266) |
| Antisense strand siRNA: GUGCACCUCUGCCAGACUGtt | (SEQ ID NO. 267) |
| Target sequence 25: AAGGAGCCTCGAGAACTGTTT | (SEQ ID NO. 268) |
| Position in gene sequence: 1111 | |
| GC content: 47.6% | |
| Sense strand siRNA: GGAGCCUCGAGAACUGUUUtt | (SEQ ID NO. 269) |
| Antisense strand siRNA: AAACAGUUCUCGAGGCUCCtt | (SEQ ID NO. 270) |
| Target sequence 26: AACTGTTTGTGAAGGGCACCA | (SEQ ID NO. 271) |
| Position in gene sequence: 1124 | |
| GC content: 47.6% | |
| Sense strand siRNA: CUGUUUGUGAAGGGCACCAtt | (SEQ ID NO. 272) |
| Antisense strand siRNA: UGGUGCCCUUCACAAACAGtt | (SEQ ID NO. 273) |
| Target sequence 27: AAGGGCACCATGAAGGACATT | (SEQ ID NO. 274) |
| Position in gene sequence: 1135 | |
| GC content: 47.6% | |
| Sense strand siRNA: GGGCACCAUGAAGGACAUUtt | (SEQ ID NO. 275) |
| Antisense strand siRNA: AAUGUCCUUCAUGGUGCCCtt | (SEQ ID NO. 276) |
| Target sequence 28: AAGGACATTCGTGAGAACTTC | (SEQ ID NO. 277) |
| Position in gene sequence: 1147 | |
| GC content: 42.9% | |
| Sense strand siRNA: GGACAUUCGUGAGAACUUCtt | (SEQ ID NO. 278) |
| Antisense strand siRNA: GAAGUUCUCACGAAUGUCCtt | (SEQ ID NO. 279) |
| Target sequence 29: AACTTCCAGGACCTGATGCAG | (SEQ ID NO. 280) |
| Position in gene sequence: 1162 | |
| GC content: 52.4% | |
| Sense strand siRNA: CUUCCAGGACCUGAUGCAGtt | (SEQ ID NO. 281) |
| Antisense strand siRNA: CUGCAUCAGGUCCUGGAAGtt | (SEQ ID NO. 282) |
| Target sequence 30: AACCAGAACTGGGAGCGTTAC | (SEQ ID NO. 283) |
| Position in gene sequence: 1312 | |
| GC content: 52.4% | |
| Sense strand siRNA: CCAGAACUGGGAGCGUUACtt | (SEQ ID NO. 284) |
| Antisense strand siRNA: GUAACGCUCCCAGUUCUGGtt | (SEQ ID NO. 285) |
| Target sequence 31: AACTGGGAGCGTTACCTGGCA | (SEQ ID NO. 286) |
| Position in gene sequence: 1318 | |
| GC content: 57.1% | |
| Sense strand siRNA: CUGGGAGCGUUACCUGGCAtt | (SEQ ID NO. 287) |
| Antisense strand siRNA: UGCCAGGUAACGCUCCCAGtt | (SEQ ID NO. |
| | (SEQ ID NO. 288) |
| Target sequence 32: AAGAAGTCGTTGATGGATCTG | (SEQ ID NO. 289) |
| Position in gene sequence: 1378 | |
| GC content: 42.9% | |
| Sense strand siRNA: GAAGUCGUUGAUGGAUCUGtt | (SEQ ID NO. 290) |
| Antisense strand siRNA: CAGAUCCAUCAACGACUUCtt | (SEQ ID NO. 291) |
| Target sequence 33: AAGTCGTTGATGGATCTGGCC | (SEQ ID NO. 292) |
| Position in gene sequence: 1381 | |
| GC content: 52.4% | |
| Sense strand siRNA: GUCGUUGAUGGAUCUGGCCtt | (SEQ ID NO. 293) |
| Antisense strand siRNA: GGCCAGAUCCAUCAACGACtt | (SEQ ID NO. 294) |
| Target sequence 34: AATGATGCCTGCCAGCTGCTC | (SEQ ID NO. 295) |
| Position in gene sequence: 1402 | |
| GC content: 57.1% | |
| Sense strand siRNA: UGAUGCCUGCCAGCUGCUCtt | (SEQ ID NO. 296) |
| Antisense strand siRNA: GAGCAGCUGGCAGGCAUCAtt | (SEQ ID NO. 297) |
| Target sequence 35: AAAGAAGACCCCTGGCTCTGG | (SEQ ID NO. 298) |
| Position in gene sequence: 1438 | |
| GC content: 57.1% | |
| Sense strand siRNA: AGAAGACCCCUGGCUCUGGtt | (SEQ ID NO. 299) |
| Antisense strand siRNA: CCAGAGCCAGGGGUCUUCUtt | (SEQ ID NO. 300) |
| Target sequence 36: AAGACCCCTGGCTCTGGGACC | (SEQ ID NO. 301) |
| Position in gene sequence: 1442 | |
| GC content: 66.7% | |
| Sense strand siRNA: GACCCCUGGCUCUGGGACCtt | (SEQ ID NO. 302) |
| Antisense strand siRNA: GGUCCCAGAGCCAGGGGUCtt | (SEQ ID NO. 303) |
| Target sequence 37: AAGAATTTAAGCAGAAGAAAG | (SEQ ID NO. 304) |
| Position in gene sequence: 1478 | |
| GC content: 28.6% | |
| Sense strand siRNA: GAAUUUAAGCAGAAGAAAGtt | (SEQ ID NO. 305) |
| Antisense strand siRNA: CUUUCUUCUGCUUAAAUUCtt | (SEQ ID NO. 306) |
| Target sequence 38: AATTTAAGCAGAAGAAAGCTA | (SEQ ID NO. 307) |
| Position in gene sequence: 1481 | |
| GC content: 28.6% | |
| Sense strand siRNA: UUUAAGCAGAAGAAAGCUAtt | (SEQ ID NO. 308) |
| Antisense strand siRNA: UAGCUUUCUUCUGCUUAAAtt | (SEQ ID NO. 309) |
| Target sequence 39: AAGCAGAAGAAAGCTAAGAAG | (SEQ ID NO. 310) |
| Position in gene sequence: 1486 | |
| GC content: 38.1% | |
| Sense strand siRNA: GCAGAAGAAAGCUAAGAAGtt | (SEQ ID NO. 311) |
| Antisense strand siRNA: CUUCUUAGCUUUCUUCUGCtt | (SEQ ID NO. 312) |
| Target sequence 40: AAGAAAGCTAAGAAGGTGAAG | (SEQ ID NO. 313) |
| Position in gene sequence: 1492 | |
| GC content: 38.1% | |
| Sense strand siRNA: GAAAGCUAAGAAGGUGAAGtt | (SEQ ID NO. 314) |
| Antisense strand siRNA: CUUCACCUUCUUAGCUUUCtt | (SEQ ID NO. 315) |
| Target sequence 41: AAAGCTAAGAAGGTGAAGAAG | (SEQ ID NO. 316) |
| Position in gene sequence: 1495 | |
| GC content: 38.1% | |
| Sense strand siRNA: AGCUAAGAAGGUGAAGAAGtt | (SEQ ID NO. 317) |
| Antisense strand siRNA: CUUCUUCACCUUCUUAGCUtt | (SEQ ID NO. 318) |
| Target sequence 42: AAGAAGGTGAAGAAGGAACCA | (SEQ ID NO. 319) |
| Position in gene sequence: 1501 | |
| GC content: 42.9% | |
| Sense strand siRNA: GAAGGUGAAGAAGGAACCAtt | (SEQ ID NO. 320) |
| Antisense strand siRNA: UGGUUCCUUCUUCACCUUCtt | (SEQ ID NO. 321) |
| Target sequence 43: AAGGTGAAGAAGGAACCAGCC | (SEQ ID NO. 322) |
| Position in gene sequence: 1504 | |
| GC content: 52.4% | |
| Sense strand siRNA: GGUGAAGAAGGAACCAGCCtt | (SEQ ID NO. 323) |
| Antisense strand siRNA: GGCUGGUUCCUUCUUCACCtt | (SEQ ID NO. 324) |
| Target sequence 44: AAGAAGGAACCAGCCACAGCC | (SEQ ID NO. 325) |
| Position in gene sequence: 1510 | |
| GC content: 57.1% | |
| Sense strand siRNA: GAAGGAACCAGCCACAGCCtt | (SEQ ID NO. 326) |
| Antisense strand siRNA: GGCUGUGGCUGGUUCCUUCtt | (SEQ ID NO. 327) |
| Target sequence 45: AAGGAACCAGCCACAGCCAGC | (SEQ ID NO. 328) |
| Position in gene sequence: 1513 | |
| GC content: 61.9% | |
| Sense strand siRNA: GGAACCAGCCACAGCCAGCtt | (SEQ ID NO. 329) |
| Antisense strand siRNA: GCUGGCUGUGGCUGGUUCCtt | (SEQ ID NO. 330) |
| Target sequence 46: AACCAGCCACAGCCAGCAAGT | (SEQ ID NO. 331) |
| Position in gene sequence: 1517 | |
| GC content: 57.1% | |
| Sense strand siRNA: CCAGCCACAGCCAGCAAGUtt | (SEQ ID NO. 332) |
| Antisense strand siRNA: ACUUGCUGGCUGUGGCUGGtt | (SEQ ID NO. 333) |
| Target sequence 47: AAGTTGCCCATCGAGGGGGCT | (SEQ ID NO. 334) |
| Position in gene sequence: 1534 | |
| GC content: 61.9% | |
| Sense strand siRNA: GUUGCCCAUCGAGGGGGCUtt | (SEQ ID NO. 335) |
| Antisense strand siRNA: AGCCCCCUCGAUGGGCAACtt | (SEQ ID NO. 336) |
| Target sequence 48: AAGACCTCGGCCCCTGCAGTG | (SEQ ID NO. 337) |
| Position in gene sequence: 1583 | |
| GC content: 66.7% | |
| Sense strand siRNA: GACCUCGGCCCCUGCAGUGtt | (SEQ ID NO. 338) |
| Antisense strand siRNA: CACUGCAGGGGCCGAGGUCtt | (SEQ ID NO. 339) |
| Target sequence 49: AACAAGATGTCATGGCCCGCG | (SEQ ID NO. 340) |
| Position in gene sequence: 1619 | |
| GC content: 57.1% | |
| Sense strand siRNA: CAAGAUGUCAUGGCCCGCGtt | (SEQ ID NO. 341) |
| Antisense strand siRNA: CGCGGGCCAUGACAUCUUGtt | (SEQ ID NO. 342) |
| Target sequence 50: AAGATGTCATGGCCCGCGCCT | (SEQ ID NO. 343) |
| Position in gene sequence: 1622 | |
| GC content: 61.9% | |
| Sense strand siRNA: GAUGUCAUGGCCCGCGCCUtt | (SEQ ID NO. 344) |
| Antisense strand siRNA: AGGCGCGGGCCAUGACAUCtt | (SEQ ID NO. 345) |
| Target sequence 51: AAGCTGAAGGGGACCACAGAG | (SEQ ID NO. 346) |
| Position in gene sequence: 1651 | |
| GC content: 57.1% | |
| Sense strand siRNA: GCUGAAGGGGACCACAGAGtt | (SEQ ID NO. 347) |
| Antisense strand siRNA: CUCUGUGGUCCCCUUCAGCtt | (SEQ ID NO. 348) |
| Target sequence 52: AAGGGGACCACAGAGCTCCTG | (SEQ ID NO. 349) |
| Position in gene sequence: 1657 | |
| GC content: 61.9% | |
| Sense strand siRNA: GGGGACCACAGAGCUCCUGtt | (SEQ ID NO. 350) |
| Antisense strand siRNA: CAGGAGCUCUGUGGUCCCCtt | (SEQ ID NO. 351) |
| Target sequence 53: AAGCGGCCCCAGCACCTTCCT | (SEQ ID NO. 352) |
| Position in gene sequence: 1681 | |
| GC content: 66.7% | |
| Sense strand siRNA: GCGGCCCCAGCACCUUCCUtt | (SEQ ID NO. 353) |
| Antisense strand siRNA: AGGAAGGUGCUGGGGCCGCtt | (SEQ ID NO. 354) |
| Target sequence 54: AAGCTCTGCCCCCGGCTAGAC | (SEQ ID NO. 355) |
| Position in gene sequence: 1723 | |
| GC content: 66.7% | |
| Sense strand siRNA: GCUCUGCCCCCGGCUAGACtt | (SEQ ID NO. 356) |
| Antisense strand siRNA: GUCUAGCCGGGGGCAGAGCtt | (SEQ ID NO. 357) |
| Target sequence 55: AAACTCATGGCACTTACCTGG | (SEQ ID NO. 358) |
| Position in gene sequence: 1801 | |
| GC content: 47.6% | |
| Sense strand siRNA: ACUCAUGGCACUUACCUGGtt | (SEQ ID NO. 359) |
| Antisense strand siRNA: CCAGGUAAGUGCCAUGAGUtt | (SEQ ID NO. 360) |
| Target sequence 56: AACCTGGCCAAGCTGCCGACA | (SEQ ID NO. 361) |
| Position in gene sequence: 1888 | |
| GC content: 61.9% | |
| Sense strand siRNA: CCUGGCCAAGCUGCCGACAtt | (SEQ ID NO. 362) |
| Antisense strand siRNA: UGUCGGCAGCUUGGCCAGGtt | (SEQ ID NO. 363) |
| Target sequence 57: AAGCTGCCGACAGGTACCACC | (SEQ ID NO. 364) |
| Position in gene sequence: 1897 | |
| GC content: 61.9% | |
| Sense strand siRNA: GCUGCCGACAGGUACCACCtt | (SEQ ID NO. 365) |
| Antisense strand siRNA: GGUGGUACCUGUCGGCAGCtt | (SEQ ID NO. 366) |
| Target sequence 58: AAGCACTGTCTCGAACAGGGG | (SEQ ID NO. 367) |
| Position in gene sequence: 1972 | |
| GC content: 57.1% | |
| Sense strand siRNA: GCACUGUCUGGAACAGGGGtt | (SEQ ID NO. 368) |
| Antisense strand siRNA: CCCCUGUUCGAGACAGUGCtt | (SEQ ID NO. 369) |
| Target sequence 59: AACAGGGGAAGCAGCAGCTGA | (SEQ ID NO. 370) |
| Position in gene sequence: 1985 | |
| GC content: 57.1% | |
| Sense strand siRNA: CAGGGGAAGCAGCAGCUGAtt | (SEQ ID NO. 371) |
| Antisense strand siRNA: UCAGCUGCUGCUUCCCCUGtt | (SEQ ID NO. 372) |
| Target sequence 60: AAGCAGCAGCTGATGCCCCAG | (SEQ ID NO. 373) |
| Position in gene sequence: 1993 | |
| GC content: 61.9% | |
| Sense strand siRNA: GCAGCAGCUGAUGCCCCAGtt | (SEQ ID NO. 374) |
| Antisense strand siRNA: CUGGGGCAUCAGCUGCUGCtt | (SEQ ID NO. 375) |
| Target sequence 61: AATAGTGCCATATGGCAAACG | (SEQ ID NO. 376) |
| Position in gene sequence: 2050 | |
| GC content: 42.9% | |
| Sense strand siRNA: UAGUGCCAUAUGGCAAACGtt | (SEQ ID NO. 377) |
| Antisense strand siRNA: CGUUUGCCAUAUGGCACUAtt | (SEQ ID NO. 378) |
| Target sequence 62: AAACGGTAGAAGAACTGGATT | (SEQ ID NO. 379) |
| Position in gene sequence: 2066 | |
| GC content: 38.1% | |
| Sense strand siRNA: ACGGUAGAAGAACUGGAUUtt | (SEQ ID NO. 380) |
| Antisense strand siRNA: AAUCCAGUUCUUCUACCGUtt | (SEQ ID NO. 381) |
| Target sequence 63: AAGAACTGGATTACTTAGAAG | (SEQ ID NO. 382) |
| Position in gene sequence: 2075 | |
| GC content: 33.3% | |
| Sense strand siRNA: GAACUGGAUUACUUAGAAGtt | (SEQ ID NO. 383) |
| Antisense strand siRNA: CUUCUAAGUAAUCCAGUUCtt | (SEQ ID NO. 384) |
| Target sequence 64: AACTGGATTACTTAGAAGTGG | (SEQ ID NO. 385) |
| Position in gene sequence: 2078 | |
| GC content: 38.1% | |
| Sense strand siRNA: CUGGAUUACUUAGAAGUGGtt | (SEQ ID NO. 386) |
| Antisense strand siRNA: CCACUUCUAAGUAAUCCAGtt | (SEQ ID NO. 387) |
| Target sequence 65: AAGTGGAGGCTGAGGCCAAGA | (SEQ ID NO. 388) |
| Position in gene sequence: 2093 | |
| GC content: 57.1% | |
| Sense strand siRNA: GUGGAGGCUGAGGCCAAGAtt | (SEQ ID NO. 389) |
| Antisense strand siRNA: UCUUGGCCUCAGCCUCCACtt | (SEQ ID NO. 390) |
| Target sequence 66: AAGATGGAGAACTTGCGAGCT | (SEQ ID NO. 391) |
| Position in gene sequence: 2110 | |
| GC content: 47.6% | |
| Sense strand siRNA: GAUGGAGAACUUGCGAGCUtt | (SEQ ID NO. 392) |
| Antisense strand siRNA: AGCUCGCAAGUUCUCCAUCtt | (SEQ ID NO. 393) |
| Target sequence 67: AACTTGCGAGCTGCAGTGCCA | (SEQ ID NO. 394) |
| Position in gene sequence: 2119 | |
| GC content: 57.1% | |
| Sense strand siRNA: CUUGCGAGCUGCAGUGCCAtt | (SEQ ID NO. 395) |
| Antisense strand siRNA: UGGCACUGCAGCUCGCAAGtt | (SEQ ID NO. 396) |
| Target sequence 68: AACCCCTAGCTCTGACTGCCC | (SEQ ID NO. 397) |
| Position in gene sequence: 2144 | |
| GC content: 61.9% | |
| Sense strand siRNA: CCCCUAGCUCUGACUGCCCtt | (SEQ ID NO. 398) |
| Antisense strand siRNA: GGGCAGUCAGAGCUAGGGGtt | (SEQ ID NO. 399) |
| Target sequence 69: AAGGACACCCAGCCCAGCTAT | (SEQ ID NO. 400) |
| Position in gene sequence: 2176 | |
| GC content: 57.1% | |
| Sense strand siRNA: GGACACCCAGCCCAGCUAUtt | (SEQ ID NO. 401) |
| Antisense strand siRNA: AUAGCUGGGCUGGGUGUCCtt | (SEQ ID NO. 402) |
| Target sequence 70: AATGGACCTTACAACGACGTG | (SEQ ID NO. 403) |
| Position in gene sequence: 2206 | |
| GC content: 47.6% | |
| Sense strand siRNA: UGGACCUUACAACGACGUGtt | (SEQ ID NO. 404) |
| Antisense strand siRNA: CACGUCGUUGUAAGGUCCAtt | (SEQ ID NO. 405) |
| Target sequence 71: AACGACGTGGACATCCCTGGC | (SEQ ID NO. 406) |
| Position in gene sequence: 2218 | |
| GC content: 61.9% | |
| Sense strand siRNA: CGACGUGGACAUCCCUGGCtt | (SEQ ID NO. 407) |
| Antisense strand siRNA: GCCAGGGAUGUCCACGUCGtt | (SEQ ID NO. 408) |
| Target sequence 72: AAGCTGCCTCACAAGGATGGT | (SEQ ID NO. 409) |
| Position in gene sequence: 2251 | |
| GC content: 52.4% | |
| Sense strand siRNA: GCUGCCUCACAAGGAUGGUtt | (SEQ ID NO. 410) |
| Antisense strand siRNA: ACCAUCCUUGUGAGGCAGCtt | (SEQ ID NO. 411) |
| Target sequence 73: AAGGATGGTAATAGCTGTAAT | (SEQ ID NO. 412) |
| Position in gene sequence: 2263 | |
| GC content: 33.3% | |
| Sense strand siRNA: GGAUGGUAAUAGCUGUAAUtt | (SEQ ID NO. 413) |
| Antisense strand siRNA: AUUACAGCUAUUACCAUCCtt | (SEQ ID NO. 414) |
| Target sequence 74: AATAGCTGTAATGTGGGAAGC | (SEQ ID NO. 415) |
| Position in gene sequence: 2272 | |
| GC content: 42.9% | |
| Sense strand siRNA: UAGCUGUAAUGUGGGAAGCtt | (SEQ ID NO. 416) |
| Antisense strand siRNA: GCUUCCCACAUUACAGCUAtt | (SEQ ID NO. 417) |
| Target sequence 75: AATGTGGGAAGCCCCTTTGCC | (SEQ ID NO. 418) |
| Position in gene sequence: 2281 | |
| GC content: 57.1% | |
| Sense strand siRNA: UGUGGGAAGCCCCUUUGCCtt | (SEQ ID NO. 419) |
| Antisense strand siRNA: GGCAAAGGGGCUUCCCACAtt | (SEQ ID NO. 420) |
| Target sequence 76: AAGCCCCTTTGCCAAGGACTT | (SEQ ID NO. 421) |
| Position in gene sequence: 2289 | |
| GC content: 52.4% | |
| Sense strand siRNA: GCCCCUUUGCCAAGGACUUtt | (SEQ ID NO. 422) |
| Antisense strand siRNA: AAGUCCUUGGCAAAGGGGCtt | (SEQ ID NO. 423) |
| Target sequence 77: AAGGACTTCCTGCCCAAGATG | (SEQ ID NO. 424) |
| Position in gene sequence: 2302 | |
| GC content: 52.4% | |
| Sense strand siRNA: GGACUUCCUGCCCAAGAUGtt | (SEQ ID NO. 425) |
| Antisense strand siRNA: CAUCUUGGGCAGGAAGUCCtt | (SEQ ID NO. 426) |
| Target sequence 78: AAGATGGAGGATGGCACCCTG | (SEQ ID NO. 427) |
| Position in gene sequence: 2317 | |
| GC content: 57.1% | |
| Sense strand siRNA: GAUGGAGGAUGGCACCCUGtt | (SEQ ID NO. 428) |
| Antisense strand siRNA: CAGGGUGCCAUCCUCCAUCtt | (SEQ ID NO. 429) |
| Target sequence 79: AAATCAACAAAATGATTTCTT | (SEQ ID NO. 430) |
| Position in gene sequence: 2378 | |
| GC content: 19% | |
| Sense strand siRNA: AUCAACAAAAUGAUUUCUUtt | (SEQ ID NO. 431) |
| Antisense strand siRNA: AAGAAAUCAUUUUGUUGAUtt | (SEQ ID NO. 432) |
| Target sequence 80: AACAAAATGATTTCTTTCTGG | (SEQ ID NO. 433) |
| Position in gene sequence: 2383 | |
| GC content: 28.6% | |
| Sense strand siRNA: CAAAAUGAUUUCUUUCUGGtt | (SEQ ID NO. 434) |
| Antisense strand siRNA: CCAGAAAGAAAUCAUUUUGtt | (SEQ ID NO. 435) |
| Target sequence 81: AAAATGATTTCTTTCTGGAGG | (SEQ ID NO. 436) |
| Position in gene sequence: 2386 | |
| GC content: 33.3% | |
| Sense strand siRNA: AAUGAUUUCUUUCUGGAGGtt | (SEQ ID NO. 437) |
| Antisense strand siRNA: CCUCCAGAAAGAAAUCAUUtt | (SEQ ID NO. 438) |
| Target sequence 82: AATGATTTCTTTCTGGAGGAA | (SEQ ID NO. 439) |
| Position in gene sequence: 2388 | |
| GC content: 33.3% | |
| Sense strand siRNA: UGAUUUCUUUCUGGAGGAAtt | (SEQ ID NO. 440) |
| Antisense strand siRNA: UUCCUCCAGAAAGAAAUCAtt | (SEQ ID NO. 441) |
| Target sequence 83: AACGCCCATAAACGTATCAGC | (SEQ ID NO. 442) |
| Position in gene sequence: 2407 | |
| GC content: 47.6% | |
| Sense strand siRNA: CGCCCAUAAACGUAUCAGCtt | (SEQ ID NO. 443) |
| Antisense strand siRNA: GCUGAUACGUUUAUGGGCGtt | (SEQ ID NO. 444) |
| Target sequence 84: AAACGTATCAGCTCCCAGATG | (SEQ ID NO. 445) |
| Position in gene sequence: 2416 | |
| GC content: 47.6% | |
| Sense strand siRNA: ACGUAUCAGCUCCCAGAUGtt | (SEQ ID NO. 446) |
| Antisense strand siRNA: CAUCUGGGAGCUGAUACGUtt | (SEQ ID NO. 447) |
| Target sequence 85: AAGGCCTCTATGGGGCCATCC | (SEQ ID NO. 448) |
| Position in gene sequence: 2501 | |
| GC content: 61.9% | |
| Sense strand siRNA: GGCCUCUAUGGGGCCAUCCtt | (SEQ ID NO. 449) |
| Antisense strand siRNA: GGAUGGCCCCAUAGAGGCCtt | (SEQ ID NO. 450) |
| Target sequence 86: AAGTGGTGACTGCCGGCACCA | (SEQ ID NO. 451) |
| Position in gene sequence: 2528 | |
| GC content: 61.9% | |
| Sense strand siRNA: GUGGUGACUGCCGGCACCAtt | (SEQ ID NO. 452) |
| Antisense strand siRNA: UGGUGCCGGCAGUCACCACtt | (SEQ ID NO. 453) |
| Target sequence 87: AATGCCCGGCCTGACCGAGTA | (SEQ ID NO. 454) |
| Position in gene sequence: 2590 | |
| GC content: 61.9% | |
| Sense strand siRNA: UGCCCGGCCUGACCGAGUAtt | (SEQ ID NO. 455) |
| Antisense strand siRNA: UACUCGGUCAGGCCGGGCAtt | (SEQ ID NO. 456) |
| Target sequence 88: AAAGCCATGGTGCAGGCCCCA | (SEQ ID NO. 457) |
| Position in gene sequence: 2623 | |
| GC content: 61.9% | |
| Sense strand siRNA: AGCCAUGGUGCAGGCCCCAtt | (SEQ ID NO. 458) |
| Antisense strand siRNA: UGGGGCCUGCACCAUGGCUtt | (SEQ ID NO. 459) |
| Target sequence 89: AAGAGCTGTGGATTGCAGCTG | (SEQ ID NO. 460) |
| Position in gene sequence: 2681 | |
| GC content: 52.4% | |
| Sense strand siRNA: GAGCUGUGGAUUGCAGCUGtt | (SEQ ID NO. 461) |
| Antisense strand siRNA: CAGCUGCAAUCCACAGCUCtt | (SEQ ID NO. 462) |
| Target sequence 90: AAGAGCAGGGGCACTGATCTA | (SEQ ID NO. 463) |
| Position in gene sequence: 2773 | |
| GC content: 52.4% | |
| Sense strand siRNA: GAGCAGGGGCACUGAUCUAtt | (SEQ ID NO. 464) |
| Antisense strand siRNA: UAGAUCAGUGCCCCUGCUCtt | (SEQ ID NO. 465) |
| Target sequence 91: AAGACAGCCACTACTGTGGGC | (SEQ ID NO. 466) |
| Position in gene sequence: 2800 | |
| GC content: 57.1% | |
| Sense strand siRNA: GACAGCCACUACUGUGGGCtt | (SEQ ID NO. 467) |
| Antisense strand siRNA: GCCCACAGUAGUGGCUGUCtt | (SEQ ID NO. 468) |
| Target sequence 92: AAAATCTTCAACTACGGCCGC | (SEQ ID NO. 469) |
| Position in gene sequence: 2839 | |
| GC content: 47.6% | |
| Sense strand siRNA: AAUCUUCAACUACGGCCGCtt | (SEQ ID NO. 470) |
| Antisense strand siRNA: GCGGCCGUAGUUGAAGAUUtt | (SEQ ID NO. 471) |
| Target sequence 93: AATCTTCAACTACGGCCGCAT | (SEQ ID NO. 472) |
| Position in gene sequence: 2841 | |
| GC content: 47.6% | |
| Sense strand siRNA: UCUUCAACUACGGCCGCAUtt | (SEQ ID NO. 473) |
| Antisense strand siRNA: AUGCGGCCGUAGUUGAAGAtt | (SEQ ID NO. 474) |
| Target sequence 94: AACTACGGCCGCATCTATGGT | (SEQ ID NO. 475) |
| Position in gene sequence: 2848 | |
| GC content: 52.4% | |
| Sense strand siRNA: CUACGGCCGCAUCUAUGGUtt | (SEQ ID NO. 476) |
| Antisense strand siRNA: ACCAUAGAUGCGGCCGUAGtt | (SEQ ID NO. 477) |
| Target sequence 95: AATGCAGTTTAACCACCGGCT | (SEQ ID NO. 478) |
| Position in gene sequence: 2898 | |
| GC content: 47.6% | |
| Sense strand siRNA: UGCAGUUUAACCACCGGCUtt | (SEQ ID NO. 479) |
| Antisense strand siRNA: AGCCGGUGGUUAAACUGCAtt | (SEQ ID NO. 480) |
| Target sequence 96: AACCACCGGCTCACACAGCAG | (SEQ ID NO. 481) |
| Position in gene sequence: 2908 | |
| GC content: 61.9% | |
| Sense strand siRNA: CCACCGGCUCACACAGCAGtt | (SEQ ID NO. 482) |
| Antisense strand siRNA: CUGCUGUGUGAGCCGGUGGtt | (SEQ ID NO. 483) |
| Target sequence 97: AAGGCCCAGCAGATGTACGCT | (SEQ ID NO. 484) |
| Position in gene sequence: 2941 | |
| GC content: 57.1% | |
| Sense strand siRNA: GGCCCAGCAGAUGUACGCUtt | (SEQ ID NO. 485) |
| Antisense strand siRNA: AGCGUACAUCUGCUGGGCCtt | (SEQ ID NO. 486) |
| Target sequence 98: AAGGGCCTCCGCTGGTATCGG | (SEQ ID NO. 487) |
| Position in gene sequence: 2968 | |
| GC content: 66.7% | |
| Sense strand siRNA: GGGCCUCCGCUGGUAUCGGtt | (SEQ ID NO. 488) |
| Antisense strand siRNA: CCGAUACCAGCGGAGGCCCtt | (SEQ ID NO. 489) |
| Target sequence 99: AACCTCCCAGTGGACAGGACT | (SEQ ID NO. 490) |
| Position in gene sequence: 3025 | |
| GC content: 57.1% | |
| Sense strand siRNA: CCUCCCAGUGGACAGGACUtt | (SEQ ID NO. 491) |
| Antisense strand siRNA: AGUCCUGUCCACUGGGAGGtt | (SEQ ID NO. 492) |
| Target sequence 100: AAGGTCCAGAGAGAAACTGCA | (SEQ ID NO. 493) |
| Position in gene sequence: 3079 | |
| GC content: 47.6% | |
| Sense strand siRNA: GGUCCAGAGAGAAACUGCAtt | (SEQ ID NO. 494) |
| Antisense strand siRNA: UGCAGUUUCUCUCUGGACCtt | (SEQ ID NO. 495) |
| Target sequence 101: AAACTGCAAGGAAGTCACAGT | (SEQ ID NO. 496) |
| Position in gene sequence: 3092 | |
| GC content: 42.9% | |
| Sense strand siRNA: ACUGCAAGGAAGUCACAGUtt | (SEQ ID NO. 497) |
| Antisense strand siRNA: ACUGUGACUUCCUUGCAGUtt | (SEQ ID NO. 498) |
| Target sequence 102: AAGGAAGTCACAGTGGAAGAA | (SEQ ID NO. 499) |
| Position in gene sequence: 3099 | |
| GC content: 42.9% | |
| Sense strand siRNA: GGAAGUCACAGUGGAAGAAtt | (SEQ ID NO. 500) |
| Antisense strand siRNA: UUCUUCCACUGUGACUUCCtt | (SEQ ID NO. 501) |
| Target sequence 103: AAGTCACAGTGGAAGAAGTGG | (SEQ ID NO. 502) |
| Position in gene sequence: 3103 | |
| GC content: 47.6% | |
| Sense strand siRNA: GUCACAGUGGAAGAAGUGGtt | (SEQ ID NO. 503) |
| Antisense strand siRNA: CCACUUCUUCCACUGUGACtt | (SEQ ID NO. 504) |
| Target sequence 104: AAGAAGTGGGAGGTGGTTGCT | (SEQ ID NO. 505) |
| Position in gene sequence: 3115 | |
| GC content: 52.4% | |
| Sense strand siRNA: GAAGUGGGAGGUGGUUGCUtt | (SEQ ID NO. 506) |
| Antisense strand siRNA: AGCAACCACCUCCCACUUCtt | (SEQ ID NO. 507) |
| Target sequence 105: AAGTGGGAGGTGGTTGCTGAA | (SEQ ID NO. 508) |
| Position in gene sequence: 3118 | |
| GC content: 52.4% | |
| Sense strand siRNA: GUGGGAGGUGGUUGCUGAAtt | (SEQ ID NO. 509) |
| Antisense strand siRNA: UUCAGCAACCACCUCCCACtt | (SEQ ID NO. 510) |
| Target sequence 106: AACGGGCATGGAAGGGGGGCA | (SEQ ID NO. 511) |
| Position in gene sequence: 3137 | |
| GC content: 66.7% | |
| Sense strand siRNA: CGGGCAUGGAAGGGGGGCAtt | (SEQ ID NO. 512) |
| Antisense strand siRNA: UGCCCCCCUUCCAUGCCCGtt | (SEQ ID NO. 513) |
| Target sequence 107: AAGGGGGGCACAGAGTCAGAA | (SEQ ID NO. 514) |
| Position in gene sequence: 3148 | |
| GC content: 57.1% | |
| Sense strand siRNA: GGGGGGCACAGAGUCAGAAtt | (SEQ ID NO. 515) |
| Antisense strand siRNA: UUCUGACUCUGUGCCCCCCtt | (SEQ ID NO. 516) |
| Target sequence 108: AAATGTTCAATAAGCTTGAGA | (SEQ ID NO. 517) |
| Position in gene sequence: 3167 | |
| GC content: 28.6% | |
| Sense strand siRNA: AUGUUCAAUAAGCUUGAGAtt | (SEQ ID NO. 518) |
| Antisense strand siRNA: UCUCAAGCUUAUUGAACAUtt | (SEQ ID NO. 519) |
| Target sequence 109: AATAAGCTTGAGAGCATTGCT | (SEQ ID NO. 520) |
| Position in gene sequence: 3175 | |
| GC content: 38.1% | |
| Sense strand siRNA: UAAGCUUGAGAGCAUUGCUtt | (SEQ ID NO. 521) |
| Antisense strand siRNA: AGCAAUGCUCUCAAGCUUAtt | (SEQ ID NO. 522) |
| Target sequence 110: AAGCTTGAGAGCATTGCTACG | (SEQ ID NO. 523) |
| Position in gene sequence: 3178 | |
| GC content: 47.6% | |
| Sense strand siRNA: GCUUGAGAGCAUUGCUACGtt | (SEQ ID NO. 524) |
| Antisense strand siRNA: CGUAGCAAUGCUCUCAAGCtt | (SEQ ID NO. 525) |
| Target sequence 111: AAGAGTTTATGACCAGCCGTG | (SEQ ID NO. 526) |
| Position in gene sequence: 3269 | |
| GC content: 47.6% | |
| Sense strand siRNA: GAGUUUAUGACCAGCCGUGtt | (SEQ ID NO. 527) |
| Antisense strand siRNA: CACGGCUGGUCAUAAACUCtt | (SEQ ID NO. 528) |
| Target sequence 112: AATTGGGTGGTACAGAGCTCT | (SEQ ID NO. 529) |
| Position in gene sequence: 3292 | |
| GC content: 47.6% | |
| Sense strand siRNA: UUGGGUGGUACAGAGCUCUtt | (SEQ ID NO. 530) |
| Antisense strand siRNA: AGAGCUCUGUACCACCCAAtt | (SEQ ID NO. 531) |
| Target sequence 113: AAGTGGCTGTTTGAAGAGTTT | (SEQ ID NO. 532) |
| Position in gene sequence: 3349 | |
| GC content: 38.1% | |
| Sense strand siRNA: GUGGCUGUUUGAAGAGUUUtt | (SEQ ID NO. 533) |
| Antisense strand siRNA: AAACUCUUCAAACAGCCACtt | (SEQ ID NO. 534) |
| Target sequence 114: AAGAGTTTGCCATAGATGGGC | (SEQ ID NO. 535) |
| Position in gene sequence: 3362 | |
| GC content: 47.6% | |
| Sense strand siRNA: GAGUUUGCCAUAGAUGGGCtt | (SEQ ID NO. 536) |
| Antisense strand siRNA: GCCCAUCUAUGGCAAACUCtt | (SEQ ID NO. 537) |
| Target sequence 115: AACCTCTTGACCAGGTGCATG | (SEQ ID NO. 538) |
| Position in gene sequence: 3469 | |
| GC content: 52.4% | |
| Sense strand siRNA: CCUCUUGACCAGGUGCAUGtt | (SEQ ID NO. 539) |
| Antisense strand siRNA: CAUGCACCUGGUCAAGAGGtt | (SEQ ID NO. 540) |
| Target sequence 116: AAGCTGGGTCTGAATGACTTG | (SEQ ID NO. 541) |
| Position in gene sequence: 3499 | |
| GC content: 47.6% | |
| Sense strand siRNA: GCUGGGUCUGAAUGACUUGtt | (SEQ ID NO. 542) |
| Antisense strand siRNA: CAAGUCAUUCAGACCCAGCtt | (SEQ ID NO. 543) |
| Target sequence 117: AATGACTTGCCCCAGTCAGTC | (SEQ ID NO. 544) |
| Position in gene sequence: 3511 | |
| GC content: 52.4% | |
| Sense strand siRNA: UGACUUGCCCCAGUCAGUCtt | (SEQ ID NO. 545) |
| Antisense strand siRNA: GACUGACUGGGGCAAGUCAtt | (SEQ ID NO. 546) |
| Target sequence 118: AAGGAAGTGACCATGGATTGT | (SEQ ID NO. 547) |
| Position in gene sequence: 3571 | |
| GC content: 42.9% | |
| Sense strand siRNA: GGAAGUGACCAUGGAUUGUtt | (SEQ ID NO. 548) |
| Antisense strand siRNA: ACAAUCCAUGGUCACUUCCtt | (SEQ ID NO. 549) |
| Target sequence 119: AAGTGACCATGGATTGTAAAA | (SEQ ID NO. 550) |
| Position in gene sequence: 3575 | |
| GC content: 33.3% | |
| Sense strand siRNA: GUGACCAUGGAUUGUAAAAtt | (SEQ ID NO. 551) |
| Antisense strand siRNA: UUUUACAAUCCAUGGUCACtt | (SEQ ID NO. 552) |
| Target sequence 120: AAAACCCCTTCCAACCCAACT | (SEQ ID NO. 553) |
| Position in gene sequence: 3592 | |
| GC content: 47.6% | |
| Sense strand siRNA: AACCCCUUCCAACCCAACUtt | (SEQ ID NO. 554) |
| Antisense strand siRNA: AGUUGGGUUGGAAGGGGUUtt | (SEQ ID NO. 555) |
| Target sequence 121: AACCCCTTCCAACCCAACTGG | (SEQ ID NO. 556) |
| Position in gene sequence: 3594 | |
| GC content: 57.1% | |
| Sense strand siRNA: CCCCUUCCAACCCAACUGGtt | (SEQ ID NO. 557) |
| Antisense strand siRNA: CCAGUUGGGUUGGAAGGGGtt | (SEQ ID NO. 558) |
| Target sequence 122: AACCCAACTGGGATGGAAAGG | (SEQ ID NO. 559) |
| Position in gene sequence: 3604 | |
| GC content: 52.4% | |
| Sense strand siRNA: CCCAACUGGGAUGGAAAGGtt | (SEQ ID NO. 560) |
| Antisense strand siRNA: CCUUUCCAUCCCAGUUGGGtt | (SEQ ID NO. 561) |
| Target sequence 123: AACTGGGATGGAAAGGAGATA | (SEQ ID NO. 562) |
| Position in gene sequence: 3609 | |
| GC content: 42.9% | |
| Sense strand siRNA: CUGGGAUGGAAAGGAGAUAtt | (SEQ ID NO. 563) |
| Antisense strand siRNA: UAUCUCCUUUCCAUCCCAGtt | (SEQ ID NO. 564) |
| Target sequence 124: AAAGGAGATACGGGATTCCCC | (SEQ ID NO. 565) |
| Position in gene sequence: 3620 | |
| GC content: 52.4% | |
| Sense strand siRNA: AGGAGAUACGGGAUUCCCCtt | (SEQ ID NO. 566) |
| Antisense strand siRNA: GGGGAAUCCCGUAUCUCCUtt | (SEQ ID NO. 567) |
| Target sequence 125: AAGCGCTGGATATTTACCAGA | (SEQ ID NO. 568) |
| Position in gene sequence: 3647 | |
| GC content: 42.9% | |
| Sense strand siRNA: GCGCUGGAUAUUUACCAGAtt | (SEQ ID NO. 569) |
| Antisense strand siRNA: UCUGGUAAAUAUCCAGCGCtt | (SEQ ID NO. 570) |
| Target sequence 126: AATTGAACTCACCAAAGGCTC | (SEQ ID NO. 571) |
| Position in gene sequence: 3669 | |
| GC content: 42.9% | |
| Sense strand siRNA: UUGAACUCACCAAAGGCUCtt | (SEQ ID NO. 572) |
| Antisense strand siRNA: GAGCCUUUGGUGAGUUCAAtt | (SEQ ID NO. 573) |
| Target sequence 127: AACTCACCAAAGGCTCCTTGG | (SEQ ID NO. 574) |
| Position in gene sequence: 3674 | |
| GC content: 52.4% | |
| Sense strand siRNA: CUCACCAAAGGCUCCUUGGtt | (SEQ ID NO. 575) |
| Antisense strand siRNA: CCAAGGAGCCUUUGGUGAGtt | (SEQ ID NO. 576) |
| Target sequence 128: AAAGGCTCCTTGGAAAAACGA | (SEQ ID NO. 577) |
| Position in gene sequence: 3682 | |
| GC content: 42.9% | |
| Sense strand siRNA: AGGCUCCUUGGAAAAACGAtt | (SEQ ID NO. 578) |
| Antisense strand siRNA: UCGUUUUUCCAAGGAGCCUtt | (SEQ ID NO. 579) |
| Target sequence 129: AAAAACGAAGCCAGCCTGGAC | (SEQ ID NO. 580) |
| Position in gene sequence: 3695 | |
| GC content: 52.4% | |
| Sense strand siRNA: AAACGAAGCCAGCCUGGACtt | (SEQ ID NO. 581) |
| Antisense strand siRNA: GUCCAGGCUGGCUUCGUUUtt | (SEQ ID NO. 571) |
| Target sequence 130: AAACGAAGCCAGCCTGGACCA | (SEQ ID NO. 572) |
| Position in gene sequence: 3697 | |
| GC content: 57.1% | |
| Sense strand siRNA: ACGAAGCCAGCCUGGACCAtt | (SEQ ID NO. 573) |
| Antisense strand siRNA: UGGUCCAGGCUGGCUUCGUtt | (SEQ ID NO. 574) |

1.-113. (canceled)

114. A composition comprising:
a recombinant polypeptide comprising an organelle localization signal operably linked to a protein transduction domain, wherein the recombinant polypeptide is operably linked to a polynucleotide.

115. The composition of claim 114, wherein the composition is a viral vector.

116. A composition according to claim 114, wherein the composition is a recombinant bacteriophage.

117. A composition according to claim 114, wherein the organelle localization signal operably linked to the protein transduction domain is expressed on an exterior surface of a vector.

118. A composition according to claim 114, wherein the compositions is a virus particle.

119. A composition according to claim 114, wherein the polynucleotide encodes a mitochondrial protein, a chloroplast protein, heterologous polypeptide, siRNA or antisense nucleic acid specific for mitochondrial or chloroplast mRNA.

120. A method for treating a mitochondrial disease comprising contacting at least one cell of a host having or suspected of having a mitochondrial disease with a vector comprising at least one protein transduction domain on a surface of the vector, at least one mitochondrial targeting signal on a surface of the vector, and a polynucleotide encoding a functional mitochondrial polypeptide, wherein the functional mitochondrial polypeptide is expressed in at least one mitochondrion of the cell.

121. A cell comprising a composition according to claim 114.

122. A method for producing a mtDNA depleted cell comprising: contacting the cell with at least one siRNA directed to POLγ.

123. The method of claim 122, wherein the siRNA is selected from the siRNAs listed in TABLE 3 or a sequence having 80-100% homology to the siRNAs listed in TABLE 3 or SEQ ID NOs. 195574.

124. The method of claim 123, wherein a target sequence of POLγ RNA inhibition is selected from the POLγ target sequences in TABLE 3.

125. A kit for transfecting organelles, the kit comprising: a polynucleotide encoding an organelle localization signal operably linked to a bacteriophage lambda surface polypeptide; and bacteriophage lambda packaging components for preparing a recombinant lambda vector.

126. A method for transfecting an organelle, the method comprising the step of introducing a recombinant viral vector comprising a nucleic acid to be expressed in the organelle into the cytosol of a cell, wherein the recombinant viral vector displays an organelle localization signal on a surface of the vector for directing the vector to an organelle to be transfected.

127. A method for producing a cell lacking mitochondrial DNA comprising: contacting the cell with at least one siRNA directed to a gene involved in sustaining or maintaining mtDNA or mtDNA stability.
